(19) United States
(12) Patent Application Publication
(10) Pub. No.: US 2026/0191670 A1
(43) Pub. Date: Jul. 9, 2026

(54) LIP SEALS AND TIPS FOR PROSTHETIC DIGITS

(71) Applicant: Touch Bionics Limited, Livingston (GB)

(72) Inventors: Alexander Campbell, Livingston (GB); Christopher William Pugh, Livingston (GB)

(21) Appl. No.: 19/443,878

(22) Filed: Jan. 8, 2026

Related U.S. Application Data
(60) Provisional application No. 63/743,381, filed on Jan. 9, 2025.

(51) Int. Cl. A61F 2/58; A61F 2/50

(52) U.S. Cl. CPC A61F 2/586; A61F 2002/5089

(57) ABSTRACT

A prosthetic hand having one or more digits with a lip seal and a tip, which may be a conductive tip. The lip seal has a cross-sectional profile with a contact finger inclining radially outwardly from a radially inner main body portion. The contact finger can flex to account for variable gap sizes in which the seal is used. Less motor current draw is required for digit actuation as compared to use of an O-ring seal. The lip seal can provide a waterproof seal to the knuckle. The prosthetic digit may include a replaceable, and/or conductive, distal tip. The replaceable distal tips may reversibly couple to the prosthetic digit via push on and removal with a simple tool. The conductive distal tips may harness the capacitance of metallic portions of the digit. Contact of the tip may be recognized by and operable with capacitive touch screens, such as mobile devices.

### INCORPORATION BY REFERENCE TO ANY PRIORITY APPLICATIONS

Any and all applications for which foreign or domestic priority is claimed are identified in the Application Data Sheet as filed with the present application and are hereby incorporated by reference under 37 CFR. 1.37. For example, this application claims priority to U.S. Provisional Patent Application No. 63/743,381, titled “LIP SEALS AND CONDUCTIVE TIPS FOR PROSTHETIC DIGITS”, filed on January 9, 2025, the entire content of which is incorporated herein by reference in its entirety and for all purposes.

### BACKGROUND

### Field

This disclosure relates to prosthetics, in particular to lip seals and tips for prosthetic digits, for example, for waterproof knuckle attachments and touch screen-compatible digits.

### DESCRIPTION OF THE RELATED ART

Prosthetic devices are essential tools that restore functionality and independence to individuals with limb loss. However, one of the significant challenges faced by users is the lack of sufficient sealing, such as for waterproofing, particularly at the knuckle or junctions between prosthetic digits and the palm portions of prosthetic hands. This limitation restricts users from performing everyday tasks in wet environments, such as washing dishes, swimming, or bathing. Water infiltration can damage electronic components, reduce the lifespan of the prosthetic, and compromise its functionality. Additionally, the inability to use prosthetics in water limits their utility and convenience, affecting the user’s quality of life and confidence in diverse settings. A waterproof attachment would not only enhance the durability of the prosthetic device but also expand its usability, allowing users to participate in water-related activities and maintain hygiene without fear of damaging their prosthetic.

Designing a completely waterproof prosthetic hand is challenging due to the need to seal electronics and sensors without compromising performance. Conventional solutions utilize waterproof gloves over the hand to protect the sensitive working parts and electronic components of the hand, but they are not always reliable, especially in use cases where the hand is fully submerged in water. Typical seal solutions require multiple seals and make sealing particularly difficult where moving parts are being sealed.

Other existing solutions utilize O-rings as internal seals to protect motors, controllers and other electronic components that allow the prosthetic hand to function. While O-rings are commonly used to resist water ingress in prosthetic devices, they are not a failsafe solution when used with non-static sealing interfaces, such as those between prosthetic digits and prosthetic hands or the knuckle. For an O-ring to work as an effective watertight seal it must be compressed between two contact faces and maintain a constant level of interference, which cannot be ensured in use cases involving prosthetic hands and digits, as both of the contact faces will move in the course of normal use.

Difficulties in achieving an appropriate O-ring size for a particular seal junction is another drawback. If the O-ring is too small, there will not be enough interference between the O-ring and contact surface, meaning the joint will be free to move but it will not be watertight. Alternatively, if the O-ring is too big resulting in a large amount of interference with the contact surface, there will be a watertight seal but the movement in the joint will be massively restricted due to the increase of friction. Use of an O-ring with a consistent and correct size for a particular seal junction is not feasible due to the manufacturing tolerance of standard O-rings and the tolerance stack up of the parts where the O-ring sits. In the context of prosthetic hands, motors must be small enough to fit into the device and are often powered by batteries. Such motors are not capable of overcoming excess friction due to increased interference, and the excess current draw would rapidly drain the batteries. Existing solutions for waterproofing prosthetic hands are thus either imperfect at sealing or are a drain on the device’s power supply, negatively impacting the safety and quality of life of users.

Additionally, as the use of touch screens proliferates, for example with mobile devices, there is a need for prosthetic digits that are compatible with such devices. Existing solutions attempt to harness capacitance from the motor of the prosthetic digit. Such approaches are very labor intensive, impermanent, and run the risk of the motor interfering with the EMG signals.

Improvements to these and other drawbacks of existing solutions for seals and touch screen tips for prosthetic hands are therefore desirable.

### SUMMARY

The embodiments disclosed herein each have several aspects no single one of which is solely responsible for the disclosure’s desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for prosthetic digits.

The following disclosure describes non-limiting examples of some embodiments. Other embodiments of the disclosed systems and methods may or may not include the features described herein. Moreover, disclosed advantages and benefits can apply only to certain embodiments of the invention and should not be used to limit the disclosure.

In some aspects, the techniques described herein relate to a prosthetic hand including: a palm portion; a prosthetic digit including a proximal digit segment, the proximal digit segment including a drive housing and a support housing, respective edges of the drive housing and the support housing configured to couple together to form the proximal digit segment; a knuckle attachment portion configured to couple with the proximal prosthetic digit segment and the palm portion, the knuckle attachment portion including: a lip seal having a cross-sectional shape including a main body portion and a contact finger extending away from the main body portion; a knuckle gearbox; and a bearing shaft; and an axis defined by the drive housing, the knuckle attachment portion, and the support housing, where the bearing shaft is configured to extend along the axis to couple the drive housing and the support housing to the knuckle gearbox, and where the lip seal is configured to provide a waterproof seal between the knuckle gearbox and the drive housing.

In some aspects, the techniques described herein relate to a prosthetic hand, where the contact finger includes a plateaued portion and a wiper tip (wiper corner).

In some aspects, the techniques described herein relate to a prosthetic hand, where the contact finger is configured to deflect toward the main body portion.

In some aspects, the techniques described herein relate to a prosthetic hand, where the contact finger is configured to deflect away from the main body portion.

In some aspects, the techniques described herein relate to a prosthetic hand, further including a waterproof cap to seal the proximal digit segment at the drive housing.

In some aspects, the techniques described herein relate to a prosthetic hand, where an end of the drive shaft includes a flat top configured to seal the proximal digit segment at the support housing.

In some aspects, the techniques described herein relate to a prosthetic hand, further including a support seal configured to provide a waterproof seal between the knuckle gearbox and the support housing.

In some aspects, the techniques described herein relate to a prosthetic hand, where the support seal is an O-ring.

In some aspects, the techniques described herein relate to a prosthetic hand, where the knuckle gearbox is configured to house a worm wheel.

In some aspects, the techniques described herein relate to a prosthetic hand, where an internal surface of the drive housing includes a protrusion defining a groove configured to receive the lip seal.

In some aspects, the techniques described herein relate to a prosthetic hand, further including a proximal digit cover disposed over the drive housing and the support housing of the proximal digit segment.

In some aspects, the techniques described herein relate to a touch-screen compatible prosthetic digit including: a proximal digit segment; a middle digit segment; a distal tip assembly including a housing and a tip portion; and a linkage assembly configured to rotatably couple the proximal digit segment, the middle digit segment, and the distal tip assembly; where a proximal end of the tip portion includes a contact tail configured to contact and harness a capacitance of a capacitive portion of the prosthetic digit to allow the tip portion to be compatible with a capacitive touch screen.

In some aspects, the techniques described herein relate to a prosthetic digit, where the middle digit segment includes the capacitive portion.

In some aspects, the techniques described herein relate to a prosthetic digit, where the linkage assembly includes the capacitive portion,

In some aspects, the techniques described herein relate to a prosthetic digit, where at least one of the proximal digit segment, the middle digit segment, and the linkage assembly are included of a conductive material.

In some aspects, the techniques described herein relate to a prosthetic digit, where at least one of the proximal digit segment, the middle digit segment, and the linkage assembly are included of metal.

In some aspects, the techniques described herein relate to a prosthetic digit, where the tip portion of the distal assembly includes a conductive elastomer.

In some aspects, the techniques described herein relate to a prosthetic digit, where the tip portion of the distal assembly includes conductive silicone.

In some aspects, the techniques described herein relate to a prosthetic digit, where the housing of the distal tip assembly includes plastic.

In some aspects, the techniques described herein relate to a prosthetic digit, further including a PCB board coupled to the contact tail, and where the capacitive portion is the PCB board.

In some aspects, the techniques described herein relate to a prosthetic hand including a prosthetic digit comprising a proximal digit segment, a knuckle gearbox rotatably coupled with the proximal digit segment about an axis of rotation, and a lip seal extending annularly around the axis of rotation between and contacting both the knuckle gearbox and the proximal digit, the lip seal having a cross-sectional shape including a main body portion and a contact finger extending radially outwardly away from the main body portion.

In some aspects, the contact finger may include a plateaued portion and a wiper corner, where the plateaued portion of the contact finger has a width greater than or equal to a width of the main body portion.

In some aspects, the contact finger may include a plateaued portion and a wiper corner, where the plateaued portion of the contact finger has a width less than a width of the main body portion.

In some aspects, the contact finger may be configured to deflect relative to the main body portion to provide a constant amount of interference between the seal and the knuckle gearbox.

In some aspects, the main body portion may include a long dimension L that extends parallel to the axis of rotation and a short dimension W that extends perpendicular relative to the axis of rotation.

In some aspects, the techniques described herein relate to a lip seal for a prosthetic digit, the lip seal including a main body portion extending annularly along a circumferential path and having a cross-sectional profile with a first dimension configured to extend parallel to an axis of rotation when assembled with the prosthetic digit, and a contact finger extending annularly along the circumferential path and having a cross-sectional profile that is inclined outwardly from the main body portion, where the contact finger is configured to deflect relative to the main body portion to provide a constant amount of interference between the seal and a rotatable component of the prosthetic digit.

In some aspects, the contact finger may include a plateaued portion, where the plateaued portion of the contact finger has a width greater than or equal to a width of the main body portion.

In some aspects, the contact finger may include a plateaued portion, where the plateaued portion of the contact finger has a width less than a width of the main body portion.

In some aspects, the contact finger and the main body portion may form a cross-sectional profile in the shape of a "V" or a canted-V.

In some aspects, the techniques described herein relate to a prosthetic digit including a proximal digit segment, a middle digit segment, a distal tip assembly including a housing and a tip portion, and a linkage assembly configured to rotatably couple the proximal digit segment, the middle digit segment, and the distal tip assembly, where a proximal end of the tip portion includes a contact tail configured to contact a portion of the prosthetic digit.

In some aspects, the techniques described herein relate to the middle digit segment and/or the linkage assembly including the portion of the prosthetic digit, where the portion of the prosthetic digit is a capacitive portion.

In some aspects, the techniques described herein relate to at least one of the proximal digit segment, the middle digit segment, and the linkage assembly including a conductive material.

In some aspects, the techniques described herein relate to the tip portion of the distal assembly including a conductive elastomer.

In some aspects, the techniques described herein relate to the prosthetic digit further including a PCB board coupled to the contact tail, where the portion of the prosthetic digit includes the PCB board, and where the portion of the prosthetic digit is a capacitive portion.

In some aspects, the techniques described herein relate to the portion of the prosthetic digit contacting the contact tail being a capacitive portion, and the tip portion may be configured to harness a capacitance of the capacitive portion to allow the tip portion to be compatible with a capacitive touch screen.

In some aspects, the techniques described herein relate to a prosthetic digit including a proximal digit segment, a middle digit segment, a distal digit segment, and a replaceable distal tip on a distal end of the distal digit segment. The distal tip includes a distal cover portion having a body defining an internal cavity, and an elastomer tip coupled to a distal end of the distal cover portion, where the distal digit segment is housed within the internal cavity of the distal cover portion, and where the replaceable distal tip is removably coupled with the distal digit segment.

In some aspects, the techniques described herein relate to the distal cover portion including a semi-rigid material and being configured to deform to a widened configuration in response to an external force applied to the distal cover portion, and return to a neutral configuration when the external force is removed.

In some aspects, the techniques described herein relate to the distal cover portion being configured to attach to a distal end of the distal digit segment.

In some aspects, the techniques described herein relate to the distal cover portion being configured to attach to the distal digit segment via a snap-on engagement.

In some aspects, the techniques described herein relate to a proximal portion of the distal cover portion including a pair of lugs, the pair of lugs having a shape complementary to the distal digit segment.

In some aspects, the techniques described herein relate to the elastomer tip including a conductive material.

In some aspects, the techniques described herein relate to the elastomer tip extending through the body of the distal cover portion into the internal cavity, where the elastomer tip contacts the distal digit segment.

In some aspects, the techniques described herein relate to the elastomer tip being configured to harness a capacitance of the distal digit segment to allow the elastomer tip to be compatible with a capacitive touch screen.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

FIG. 1 is a dorsal view of an example of a prosthetic hand.

FIG. 2 is a perspective view showing an example prosthetic digit from the prosthetic hand of FIG. 1 having a lip seal therein.

FIG. 3 is an exploded view of the prosthetic digit of FIG. 2 showing the lip seal.

FIG. 4A shows a partially exploded perspective view of the proximal digit segment and knuckle gearbox of the prosthetic digit of FIG. 2 having the lip seal.

FIG. 4B shows the exploded view of the proximal digit segment of FIG. 4A with the knuckle gearbox removed to show an example gear assembly.

FIG. 5 is a cross-section view of the knuckle gearbox and the proximal digit segment as taken along line A-A in FIG. 2, showing a cross-sectional view of an example lip seal.

FIG. 6A is a perspective view of the lip seal of the prosthetic digit of FIG. 2.

FIG. 6B is a cross-section view of the lip seal as taken along line B-B in FIG. 6A.

FIGS. 7A-C are example cross-sectional profiles for the lip seal of FIG. 2.

FIGS. 8A and 8B are a perspective view and a partially exploded view, respectively, of an example prosthetic digit having a conductive tip.

FIG. 9 is a side cross-section view of the prosthetic digit of FIG. 8A as taken along line C-C in FIG. 8A.

FIG. 10A and 10B are a perspective and a partially exploded view, respectively, of another example prosthetic digit having a replaceable tip.

FIG. 11 shows an exploded view of the distal phalange assembly and replaceable tip assembly of the prosthetic digit of FIGS. 10A and 10B.

FIGS. 12A and 12B are a partially exploded and a perspective view, respectively, showing how the distal phalange assembly attaches to the prosthetic digit in FIGS. 10A and 10B.

FIGS. 13A-D show various views of the replaceable tip assembly of the prosthetic digit of FIG. 10A. FIGS. 13A and 13B are exploded dorsal and ventral perspective views, respectively, of the replaceable tip assembly. FIG. 13C is a ventral perspective view of the replaceable tip assembly. FIG. 13D is a side, cross-section view of the replaceable tip assembly taken along the line D-D in FIG. 13C and flipped vertically.

FIGS. 14A-E illustrate a method of replacing the replaceable tip assembly of the prosthetic digit in FIGS. 10A and 10B.

FIG. 15 is a side cross-section view of the distal phalange and replaceable tip as taken along taken along the line E-E in FIG. 14B and flipped vertically.

### DETAILED DESCRIPTION

The following detailed description is directed to certain specific embodiments of the development. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments.

The present disclosure is directed to a prosthetic hand with a waterproof and touch screen compatible prosthetic digit. The prosthetic digit can include an annular lip seal with a unique cross-sectional profile that provides a waterproof knuckle seal while allowing for energy-efficient actuation of the digit. The cross-sectional profile of the lip seal can include a generally rectangular, axial main body portion and a contact finger that extends angularly to incline radially outward and away from the main body portion. The prosthetic digit can include a conductive distal tip that harnesses the capacitance of metal or other conductive portions of the digit to enable the prosthetic digit to be compatible with capacitive touch screens.

### Example Prosthetic Hand and Digit

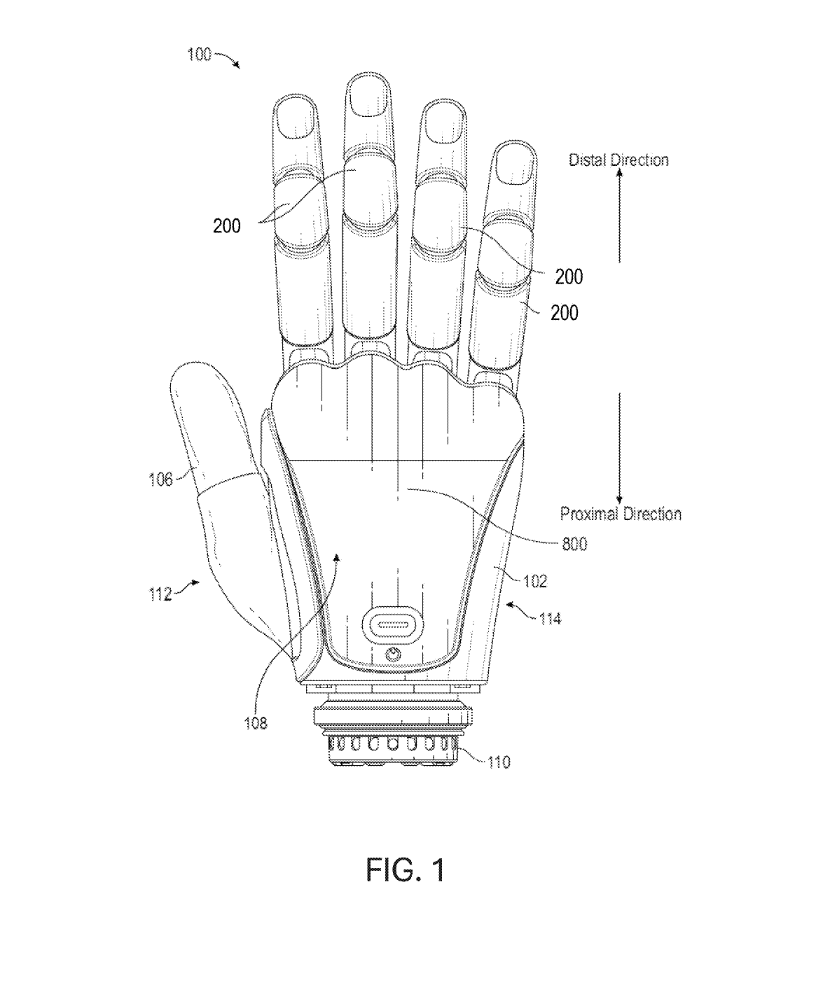

FIG. 1 is a dorsal view of an example of a prosthetic hand 100 that may be used with the prosthetic digits 200 or 400 or any other digits described herein. The prosthetic hand 100 includes four prosthetic digits 200, a prosthetic thumb 106, and a palm portion 102. The palm portion 102 includes a dorsal side 108, a palm side (not shown) opposite the dorsal side 108, a lateral inner side 112 to which the prosthetic thumb 106 is connected, and a lateral outer side 114 opposite the lateral inner side 112. A waterproof cover assembly 800 may extend over and around the palm portion 102. The prosthetic hand 100 may be attached to an upper limb such as a natural or prosthetic arm, e.g., via a wrist device 110. There may be one, two, three or four of the prosthetic digits 200. The prosthetic digits 200 are laterally spaced apart and extend distally from a distal end of the palm portion 102. The prosthetic thumb 106 extends laterally and distally from the lateral inner side 112 of the palm portion 102. Each of the prosthetic digits 200 and the thumb 106 may include one or more rotatable segments that are actuated by one or more motors and/or links to move. Each prosthetic digit 200 and the thumb 106 may be actuated by its own respective dedicated motor. Various features for prosthetic digits, thumbs, hands and wrists may be used with the prosthetic hand 100 and the various digits and tips described herein, for example those described in U.S. Pat. No. 8,986,395,“HAND PROSTHESIS”, issued on March 21, 2015, in U.S. Pat. No. 9,387,095, “PROSTHETICS AND ORTHOTICS”, issued on July 12, 2016, in U.S. Pat. No. 8,197,554, “ROTARY ACTUATOR ARRANGEMENT”, issued on June 12, 2012, in U.S. Pat. No. 9,99,522, “PROSTHETIC DIGIT FOR USE WITH TOUCHSCREEN DEVICES”, issued on June 19, 2018, in U.S. Pat. No. 11,083,600, “PROSTHETIC DIGIT FOR USE WITH TOUCHSCREEN DEVICES”, issued on August 10, 2021, in U.S. Pat. No. 10,973,660, “POWERED PROSTHETIC THUMB”, issued on April 13, 2021, in U.S. App. No. 17/199176, “POWERED PROSTHETIC THUMB”, filed on March 11, 2021, in U.S. App. No. 62/599559, “POWERED PROSTHETIC THUMB”, filed on December 15, 2017, in U.S. App. No. 17/602247, “PROSTHETIC DIGIT WITH ARTICULATING LINKS”, filed on October 7, 2021, in U.S. App. No. 62/832166, “PROSTHETIC DIGIT WITH ARTICULATING LINKS”, filed on April 10, 2019, in U.S. App. No. 17/612539, “ACTUATION SYSTEMS FOR PROSTHETIC DIGITS”, filed on November 18, 2021, in U.S. App. No. 62/850675, “ACTUATION SYSTEMS FOR PROSTHETIC DIGITS”, filed on May 21, 2019, in U.S. App. No. 17/760742, “PROSTHETIC DIGITS AND ACTUATORS”, filed on March 15, 2022, in U.S. App. No. 62/902227, “PROSTHETIC DIGIT ACTUATORS WITH GEAR SHIFTING”, filed on September 18, 2019, in U.S. App. No. 17/098045, “PROSTHETIC DIGIT ACTUATOR”, filed on November 13, 2020, in U.S. App. No. 62/935852, “PROSTHETIC DIGIT ACTUATOR”, in filed on November 15, 2019, in U.S. App. No. 63/064614, “PROSTHETIC DIGIT ACTUATOR”, filed on August 12, 2020, and/or in U.S. App. No. 18/596,285, titled “PROSTHETIC THUMB WITH ROTATION AND COMPOUND FLEXION,” and filed on March 7, 2024, in U.S. App. No. 18/596,427, “PROSTHETIC HAND WITH WATERPROOF COVER AND FILAMENT ATTACHMENT FOR DIGITS”, filed on March 5, 2024, each of which is incorporated by reference herein in its entirety and forms a part of this specification for all purposes. Thus, the various features described herein, for lip seals and/or for conductive tips, may be used with, and may incorporate a variety of features of, different prosthetic digits or thumbs.

While the prosthetic hand 100 is shown and described herein as a right-sided prosthetic hand 100, the same features may be incorporated into a mirrored, left-sided prosthetic hand, with the appropriate mirroring of the reference directions and sides as described herein for the right-sided prosthetic hand 100.

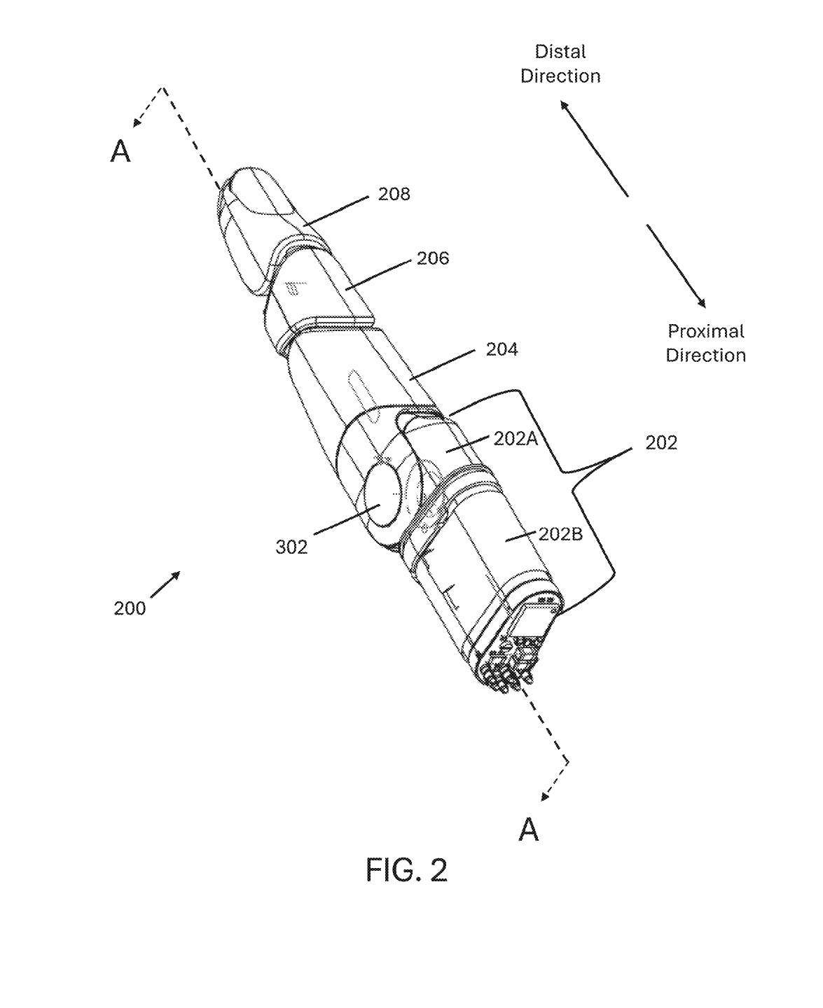

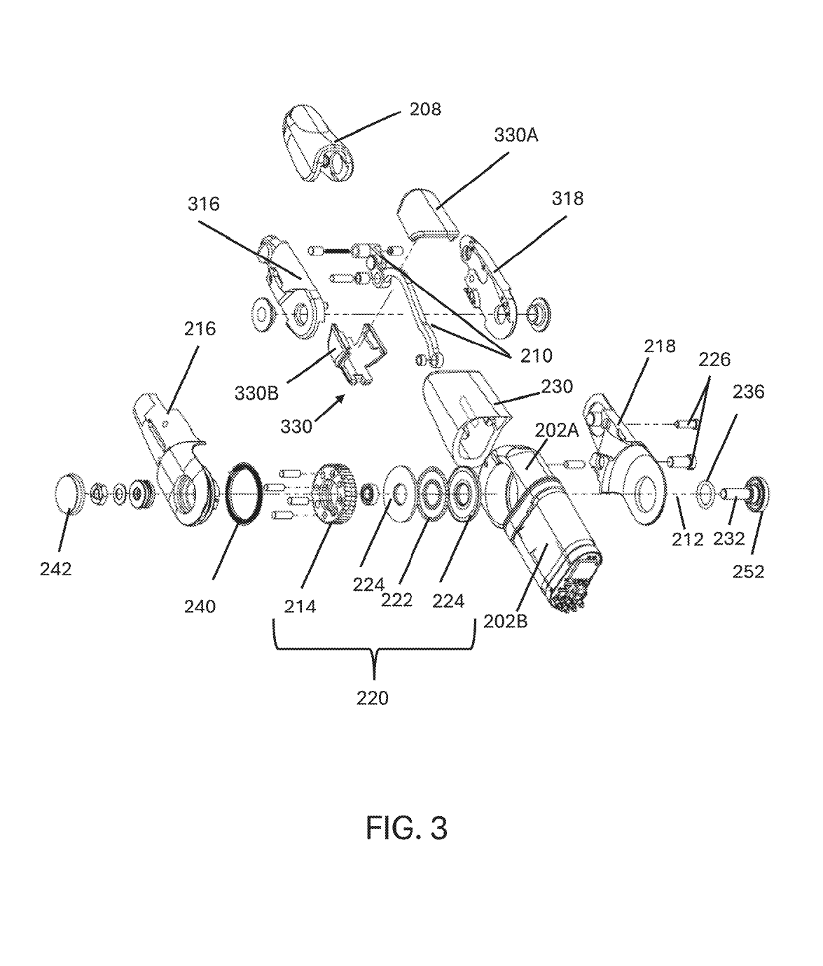

FIGS. 2 and 3 are perspective and exploded views respectively, of an example prosthetic digit 200. The prosthetic digit 200 may be used with the prosthetic hand 100. The prosthetic digit 200 may include a knuckle gearbox 202, a proximal digit segment 204, a middle digit segment 206, and/or a distal tip assembly 208. A proximal portion 202B of the knuckle gearbox 202 is attachable to a palm portion of a prosthetic hand (e.g., palm portion 102 of the prosthetic hand 100), and a distal portion 202A of the knuckle gearbox 202 is rotatably attachable to a proximal end of the proximal digit segment 204 at a knuckle attachment portion 302. The distal end of the proximal digit segment 204 is rotatably attachable to the proximal end of the middle digit segment 206. The distal end of the middle digit segment 206 is rotatably attachable to the proximal end of the distal tip assembly 208.

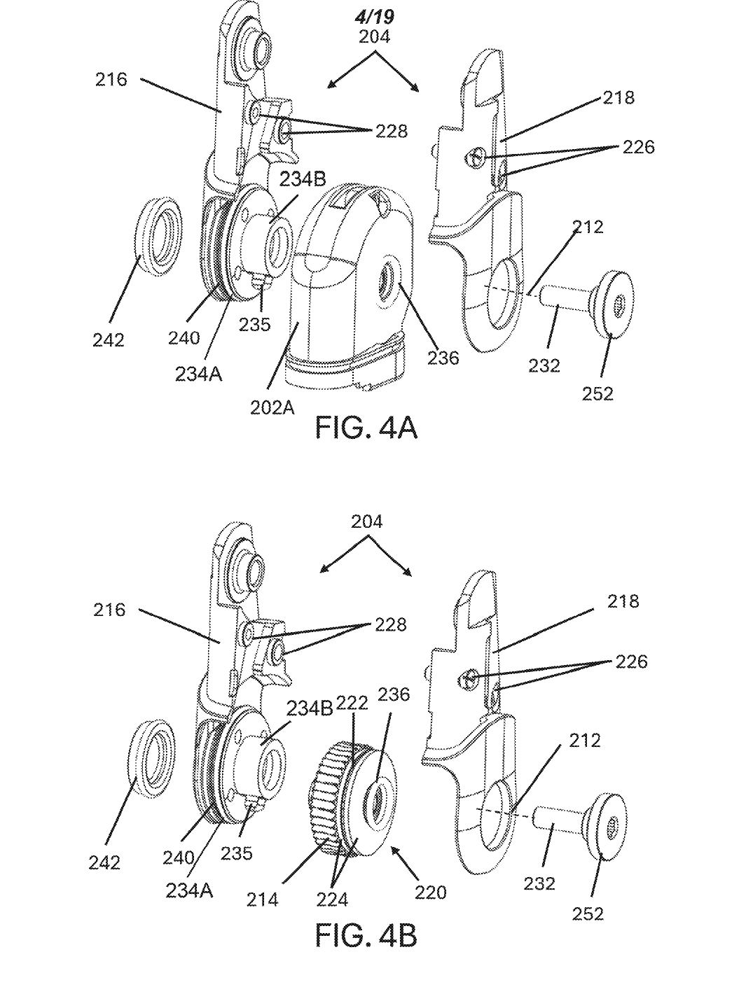

The distal portion 202A of the knuckle gearbox 202 houses a gear assembly 220. As shown in FIG. 2B, and as further described herein, for example with respect to FIGS. 4A and 4B, the gear assembly 220 can include a worm gear 214, and one or more bearings 222, 224 (e.g., radial bearings, ball bearings, race bearings thrust bearings, other bearing types, and/or combinations thereof). The worm gear 214 is mechanically coupled to a motor (not shown) housed in the proximal portion 202B of the knuckle gearbox 202, and a linkage assembly 210 housed at least partially within the body of the prosthetic digit 200. The motor causes articulation of the prosthetic digit 200. Actuation of the motor causes the worm gear 214 to rotate, which in turn rotates the proximal digit segment 204. Rotation of the proximal digit segment 204 causes rotation of the middle digit segment 206 and the distal tip assembly 208 to via the linkage assembly 210. Rotation of the worm gear 214 in a first (e.g., clockwise) direction may cause the prosthetic digit 200 to close, while rotation of the worm gear 214 in a second, opposite direction (e.g., counterclockwise) may cause the prosthetic digit 200 to open. Alternatively, rotation of the worm gear 214 in a first (e.g., clockwise) direction may cause the prosthetic digit 200 to open, while rotation of the worm gear 214 in a second, opposite direction (e.g., counterclockwise) may cause the prosthetic digit 200 to close.

The proximal digit segment 204 has a body that includes a drive housing 216 and a complementary support housing 218, which can be joined or fastened together along their respective edges (e.g., by press-fit, complementary grooves, welding, adhesives, etc.) to surround or enclose the distal portion 202A of the knuckle gearbox 202 and the gear assembly 220. When assembled, an axis 212 is defined by and extends through the drive housing 216 and complementary support housing 218, the distal portion 202A of the knuckle gearbox 202, and the gear assembly 220. An opening or channel may extend along the axis 212 defined by some or all of the aforementioned components. A bearing shaft 232 extends through this opening or channel along the axis 212 to connect together the drive housing 216 and complementary support housing 218 of the proximal digit segment 204, distal portion 202A of the knuckle gearbox 202, and the gear assembly 220. The drive housing 216 and complementary support housing 218 may additionally be secured together by one or more fasteners 226 (e.g., screws, pins, bushings, dowels, fixings, and the like, and/or combinations thereof) extending through the support housing 218, and which may be received in complementary openings 228 (see FIG. 4A) of the drive housing 216. The proximal digit segment 204 may rotate about the axis 212.

The middle digit segment 206 may have a body formed by complementary lateral housings 316, 318 which can be joined or fastened together along their respective edges (e.g., by press-fit, complementary grooves, welding, gluing, etc.). Once assembled, the lateral housings 316, 318 of the middle digit segment 206 may be surrounded by a middle segment cover 330. The middle segment cover 330 may be waterproof. The middle segment cover 300 may include a top middle digit cover 330A and a complementary bottom middle digit cover 330B that join along their respective edges around the middle digit segment 206 when the complementary lateral housings 316, 318 are joined together.

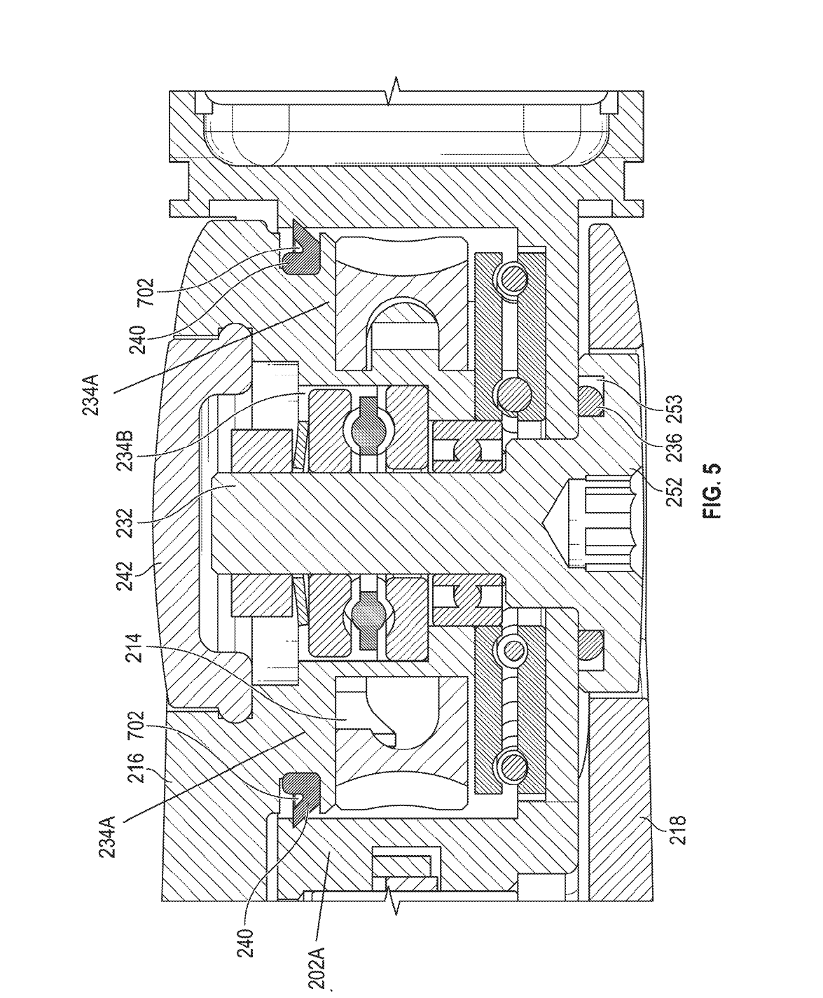

FIGS. 4A-5 show various views of the distal portion 202A of the knuckle gearbox 202 and the proximal digit segment 204. FIG. 4A shows a partially exploded perspective view of the proximal digit segment 204 and the distal portion 202A of the knuckle gearbox 202. FIG. 4B shows a partially exploded perspective view of the proximal digit segment 204 and with the body of the proximal portion 202A removed to show the gear assembly 220. FIG. 5 shows a partial cross-section view of the prosthetic digit 200 taken along line A-A as shown in FIG. 2. FIG. 5 shows the lip seal 240, and relative arrangement of the gear assembly 220, distal portion 202A of the knuckle gearbox 202 and the proximal digit segment 204 when assembled.

As discussed above, the gear assembly 220 may include the worm gear 214 with one or more ball bearings. Each ball bearing may include a series of balls 222 sandwiched between two opposing races 224. The balls 222 may be encased in a cage. A tiered protrusion 234 may extend laterally inwardly from an internal surface of the drive housing 216. The tiered protrusion 234 may include a first tier 234A and a second tier 234B extending from an inner diameter of the first tier 234B and protruding laterally inward toward the gear assembly 220. The first tier 234A may be a flange-like structure. The second tier 234B may be an annular wall. The first tier 234A and the second tier 234B of the protrusion 234 may each have a generally cylindrical or other rounded shape. The circumferential surface of the second tier 234B of the protrusion 234 may be sized to have a slightly smaller diameter than the inner diameter of the worm gear 214 to allow the worm gear 214 to slide onto the second tier 234B during assembly without impeding the ability of the worm gear 214 to rotate therein. The second tier 234B of the protrusion 234 may additionally include a stop 235 on a radially outer surface thereof. The stop 235 may restrict the range of rotation of the worm gear 214 and, consequently, the rotational range of the digit segments 204, 206 and distal tip assembly 208 such that the prosthetic digit 200 articulates like a natural digit. The first tier 234A of the protrusion 234 may define a groove for receiving the lip seal 240, as discussed in further detail herein, for example with respect to FIGS. 5-7C.

### Example Lip Seal

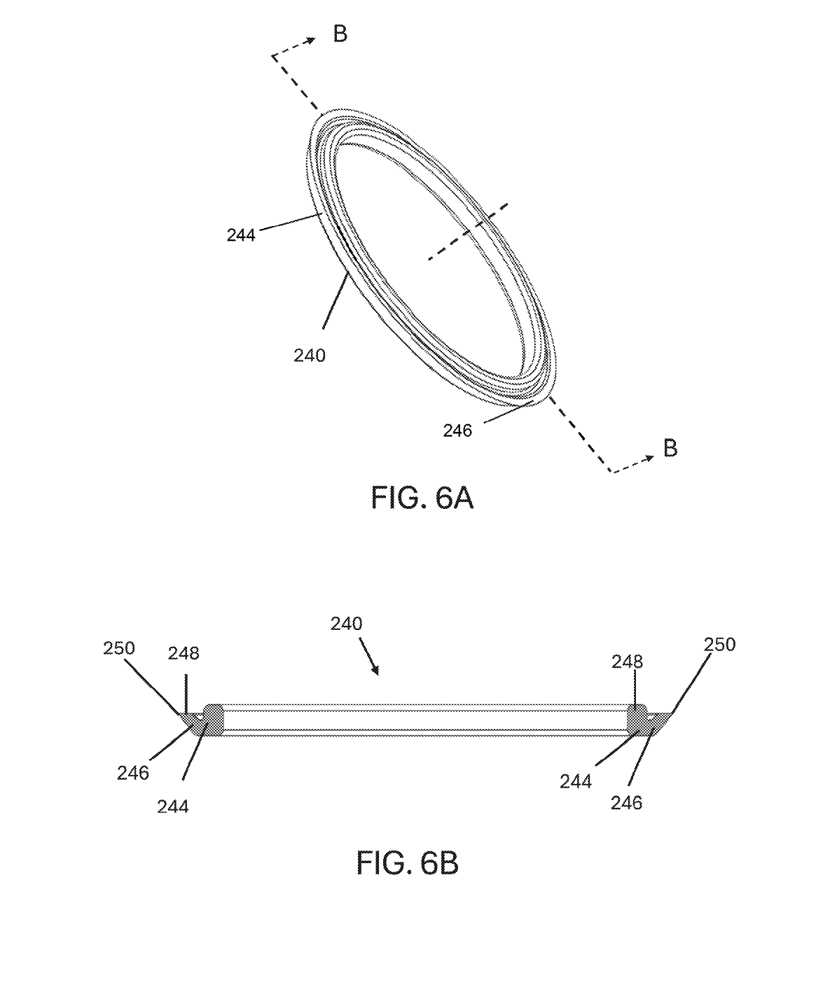

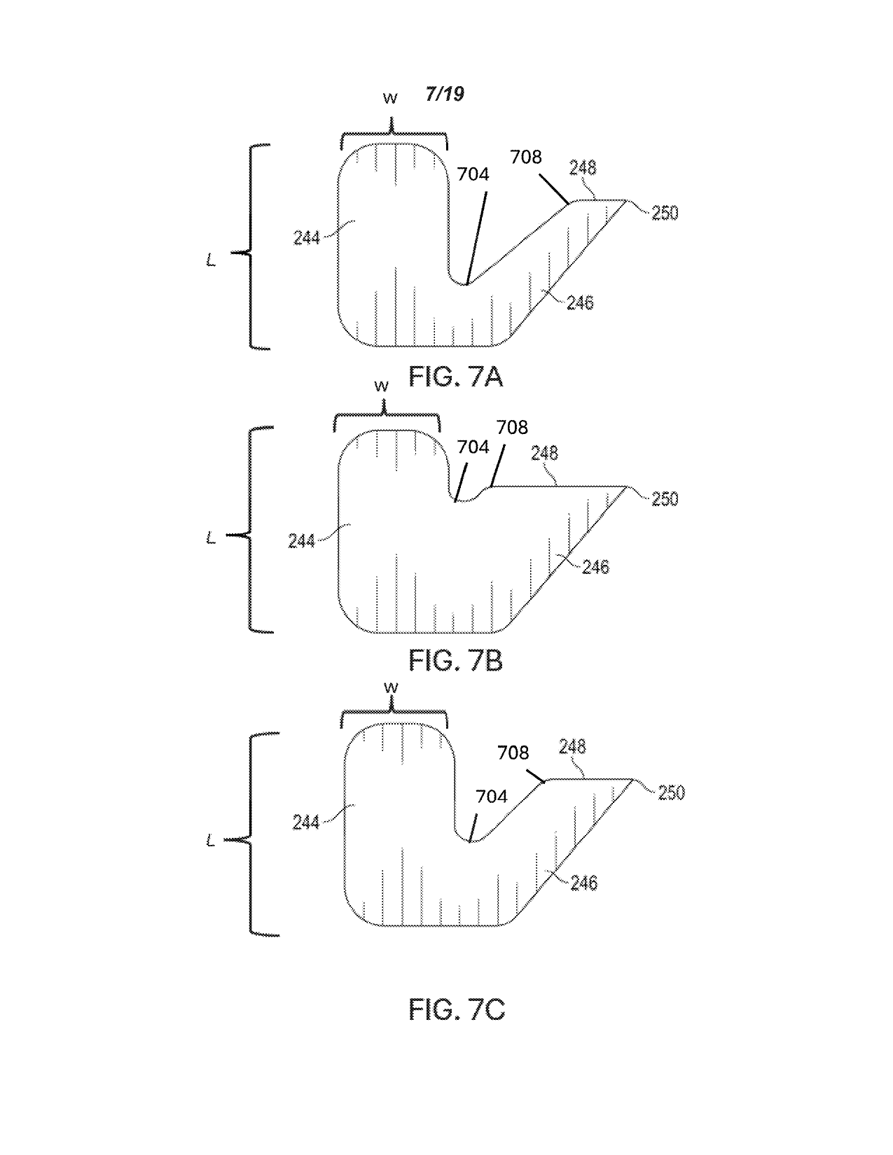

As further shown in FIGS. 4A-5, the bearing shaft 232 extends along the axis 212 and associated opening or channel through the support housing 218, the knuckle gearbox 202, gear assembly 220, and drive housing 216. The proximal digit segment 204 may be sealed on an outer lateral side of the drive housing 216 by a waterproof cap 242. The prosthetic digit 200 includes the lip seal 240 positioned between the distal portion 202A of the knuckle gearbox 202 and the drive housing 216, as discussed in further detail herein, for example with respect to FIGS. 6A-7C. The lip seal 240 may provide a combination of enhanced waterproofing and performance capabilities. The bearing shaft 232 includes a flat head 252. The outer surface of the flat head 252 seals the proximal digit segment 204 at an outer lateral side of the support housing 218. The inner surface of the flat head 252 contacts the knuckle gearbox 202 and includes a groove 253 to accommodate an additional seal 236 between the inner surface of the flat head 252 and the outer surface of the knuckle gearbox 202, to prevent water from seeping into the knuckle gearbox 202. In some embodiments, the seal 236 is an O-ring. Additionally, once the drive housing 216 and the complementary support housing 218 are assembled together, the proximal digit cover 230 (as shown in FIGS. 2 and 3) may be positioned (e.g., slid onto) the proximal digit segment 204 to cover the sealing interface between the drive housing 216 and complementary support housing 218 and help to waterproof the proximal digit segment 204.

As further shown in FIG. 5, the lip seal 240 is received in a groove defined by the first tier 234A of the protrusion 234 of the drive housing 216. When the prosthetic digit 200 is assembled, there may be a sealing gap 702 formed between the interface of the first tier 234A of the protrusion 234 of the drive housing 216 and the knuckle gearbox 202. The unique shape of the lip seal 240 allows for it to fill the sealing gap 702 and maintain a constant amount of interference between the lip seal 240 and the interface between the drive housing 216 and the knuckle gearbox 202.

FIGS. 6A and 6B show various views of the lip seal 240. FIG. 6A is a perspective view of the lip seal 240 in isolation and FIG. 6B is a cross-section view as taken along the line B-B shown in FIG. 6A.

The lip seal 240 extends annularly along a circumferential path. The lip seal 240 may be circular. The lip seal 240 may have a unique cross-sectional profile. The lip seal 240 may provide a constant or less variable amount of interference force between the seal and the sealing face(s) against which the seal contacts. The lip seal 240 may provide such advantages despite any variation in tolerance, especially as compared to traditional O-ring seals. The lip seal 240 may be formed of an elastomeric material, such as rubber or silicone.

As shown in FIGS. 6B-7C, the cross-sectional profile of the lip seal 240 may have a main body portion 244. The main body portion 244 may have a generally rectangular shape. The main body portion 244 may have rounded corners. The main body portion 244 may have a long dimension L that extends parallel to an axis of the opening extending through and defined by the annular lip seal 240. The long dimension L may extend parallel to the axis of rotation of the prosthetic digit 200. The long dimension L may extend parallel to the axis 212 when assembled with the prosthetic digit. The main body portion 244 may have a short dimension W that extends perpendicular relative to the long dimension L. The main body portion 244 may have the short dimension W that extends perpendicular relative to the axis 212 and/or the axis of rotation of the prosthetic digit 200. The long dimension L may be longer than the short dimension W. The main body portion 244 may have other shapes, such as square, trapezoidal, or triangular.

The lip seal 240 may have a lip such as a contact finger 246 extending from the main body portion 244. The contact finger 246 may extend from an end of the main body portion 244. The contact finger 246 may incline outwardly from the main body portion 244. The contact finger 246 may be canted. The contact finger 246 may extend radially outwardly and axially. The contact finger 246 and main body portion 244 may have a cross-sectional profile in the shape of a “V” or a canted-V. The contact finger 246 may have an axial outer end that is located axially lower than an upper axial end of the main body portion 244. The axial height of the contact finger 246 (e.g. as measured parallel to an axis defined by the annular lip seal 240) may be less than an axial height of the main body portion 244, for instance less than the long dimension L. The overall radial width of the profile of the lip seal 240 may be greater than the overall axial height of the lip seal 240, for example greater than the long dimension L. In some embodiments, the overall radial width of the profile of the lip seal 240 may be less than or equal to the overall axial height of the lip seal 240, for example less than or equal to the long dimension L. A transition portion 704 between the contact finger 246 and the main body portion 244 may be curved or sharp. The transition portion 704 may be adjacent a gap defined between the main body portion 244 and the contact finger 246. The transition portion 704 may be in the shape of a “U” or “V.”

The contact finger 246 may have a plateaued portion 248. The plateaued portion 248 may be a surface on an axial end of the contact finger 246. The plateaued portion 248 may be axially lower (e.g. as measured parallel to an axis defined by the annular lip seal 240) than an upper axial end of the main body portion 244. The plateaued portion 248 may extend perpendicular relative to the long dimension L of the main body portion 244. In some embodiments the plateaued portion 248 may be angled relative to a radial direction. As shown in FIGS. 7A-C, the length of the plateaued portion may vary. The corner 708 between a radially inner edge of the contact finger 246 and the plateaued portion 248 may be curved or tapered, or it may be sharp.

The contact finger 246 may have a wiper corner 250. The wiper corner 250 may be a radially outer edge of the contact finger 246. The wiper corner 250 may be on an opposite end of the plateaued portion 248 as the inner corner 708. In some embodiments, the wiper corner 250 forms a sharp, clean edge of the contact finger 246.

The contact finger 246 may extend away from the main body portion 244 to form an angle therebetween. The angle may be an acute angle (e.g., less than 90 degrees). The angle may be from 10 to 80 degrees, from 15 to 75 degrees, from 20 to 70 degrees, from 25 to 65 degrees, from 30 to 60 degrees, from 35 to 55 degrees, from 40 to 50 degrees, or 45 degrees or about 45 degrees. Such angle may be measured between geometric centerlines of the main body portion 244 and the contact finger 246. These and other features of the lip seal 240 allow for the constant amount of interference between the seal and sealing face despite any variation in tolerance stack up of the surrounding parts or of the lip seal 240 itself. For example, as shown in FIG. 5, if the sealing gap 702 between the drive housing 216 and the knuckle gearbox 202 is at the lower end of a tolerance stack (e.g. a relatively smaller gap), then the contact finger 246 can fold in on itself or deflect toward the main body portion 244, such that the wiper corner 250 extends closer to the main body portion 244. Alternatively, if the sealing gap 702 is at the higher end of the tolerance stack (e.g. a relatively larger gap), the contact finger 246 can accommodate by “opening up”, increasing the angle between the contact finger 246 and the main body portion 244 such that the wiper corner 250 is relatively farther away from the main body portion 244.

Various dimensions of the lip seal 240 may be adjusted for use in different sized gaps or to adjust the amount of sealing pressure exerted by the lip seal 240 in the gap. In some embodiments, the positional height or radial width of the plateaued portion 248 can be adjusted, and/or the thickness of the contact finger 246 may be adjusted, for example to account for applications of varying contact pressure.

FIGS. 7A-C, show additional examples of cross-sectional profiles for the lip seal 240. The contact finger 246 may have a thickness suitable for a particular application. Changing the thickness of the contact finger 246 may not affect the fit (e.g. overall radial width) of the lip seal 240 within the sealing gap 702. The thickness of the contact finger 246 may be based on a desired stiffness and sealing performance. The thickness may be uniform or variable along the length of the contact finger 246.

In the embodiment of FIG. 7A, the radial width of the plateaued portion 248 is less than the width W of main body portion 244, resulting in a contact finger 246 that is narrower at the end than the main body portion 244. The transition portion 704 in FIG. 7A is located in a lower portion of the main body portion 244. The contact finger 246 may taper down in thickness in the radially outward direction. The thickness may decrease toward the free end of the contact finger 246. The free end may have the minimal thickness of the contact finger 246. The bending stiffness of the contact finger 246 may decrease in a direction toward the free end.

In the embodiment of FIG. 7B, the radial width of the plateaued portion 248 is greater than the width W of the main body portion 244, resulting in a contact finger 246 that is wider at the end than the main body portion 244. The transition portion 704 in FIG. 7B is located in an axially upper portion of the main body portion 244. The contact finger 246 may have a constant thickness in the radially outward direction.

In the embodiment of FIG. 7C, the radial width of the plateaued portion 248 is approximately equal to the width W of the main body portion 244, resulting in an end of the contact finger 246 that has an approximately equal thickness as the main body portion 244. The transition portion 704 in FIG. 7A is located in an axially middle portion of the main body portion 244.

For the embodiments of FIGS. 7B and 7C, the thickness may be uniform toward the free end of the contact finger 246. The bending stiffness of the contact finger 246 may be the same along the length of the contact finger 246. In some embodiments, the thickness of the contact finger 246 may change, for example as described for the embodiment of FIG. 7A.

The lip seal 240 is shown to have a “V” shape or check-shaped cross-sectional profile. Other profile shapes may be implemented, such as a “U” shape with one of the upward legs inclining laterally outward as described above for the contact finger 246. In some embodiments, the lip seal 240 may have a “C” shape rotated ninety degrees to open upward and with one of the prongs of the “C” inclining laterally outward as described above for the contact finger 246. In some embodiments, the lip seal 240 may have an “L” shape with the contact finger 246 inclining laterally outward (as described above for the contact finger 246) from the lower segment of the “L”. In some embodiments, the lip seal 240 may have a “G” shape with the lower-right portion of the “G” inclining laterally outward as described above for the contact finger 246. In some embodiments, the lip seal 240 may have a “J” shape with the hook of the lower portion of the “J” inclining laterally outward as described above for the contact finger 246.

The lip seal 240 described herein advantageously allows for waterproofing the prosthetic digit. The lip seal 240 may achieve a seal with an IP67 rating. The lip seal 240 may provide such sealing for the prosthetic digit 200 at the knuckle attachment to the prosthetic hand 100, while maintaining the interference between the lip seal 240 at a low enough level to preserve the speed and battery life of the prosthetic digit 200. The lip seal 240 provides less friction relative to other seals, such as O-rings. The prosthetic digit with the lip seal 240 may thus rotate using less current draw from a motor as compared to a digit with an O-ring at a rotating interface. The prosthetic digit with the lip seal 240 may therefore rotate faster than a similar digit with an O-ring.

### Example Digit Tips

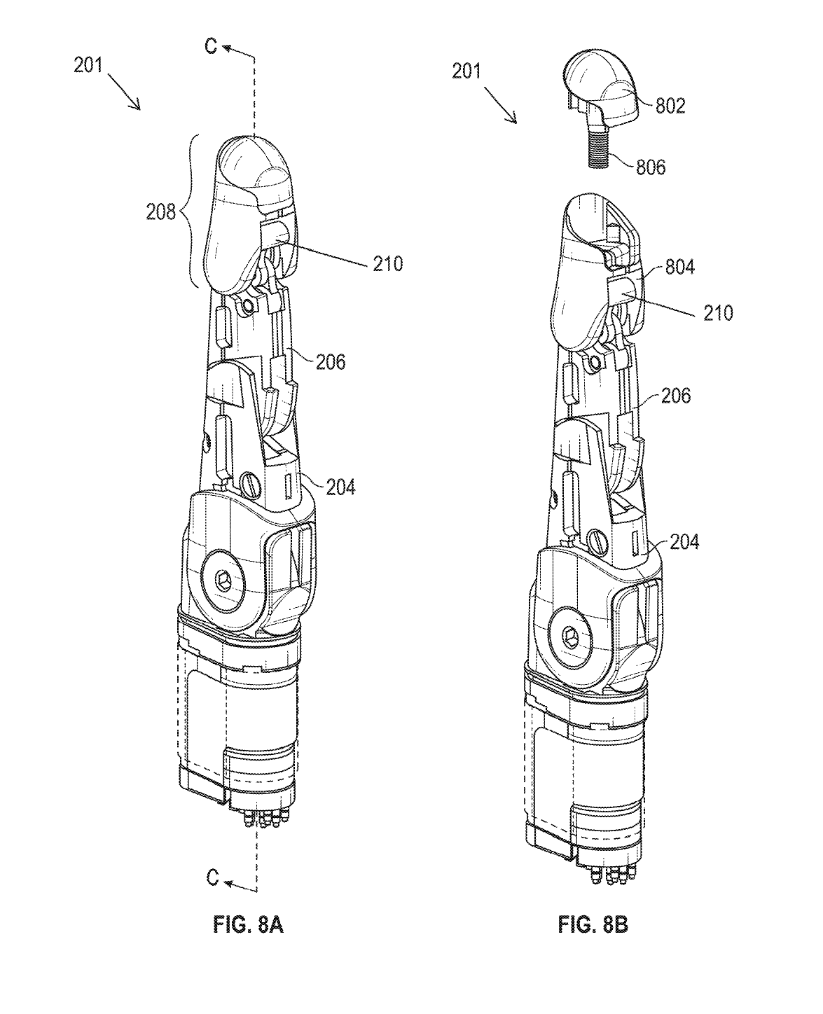

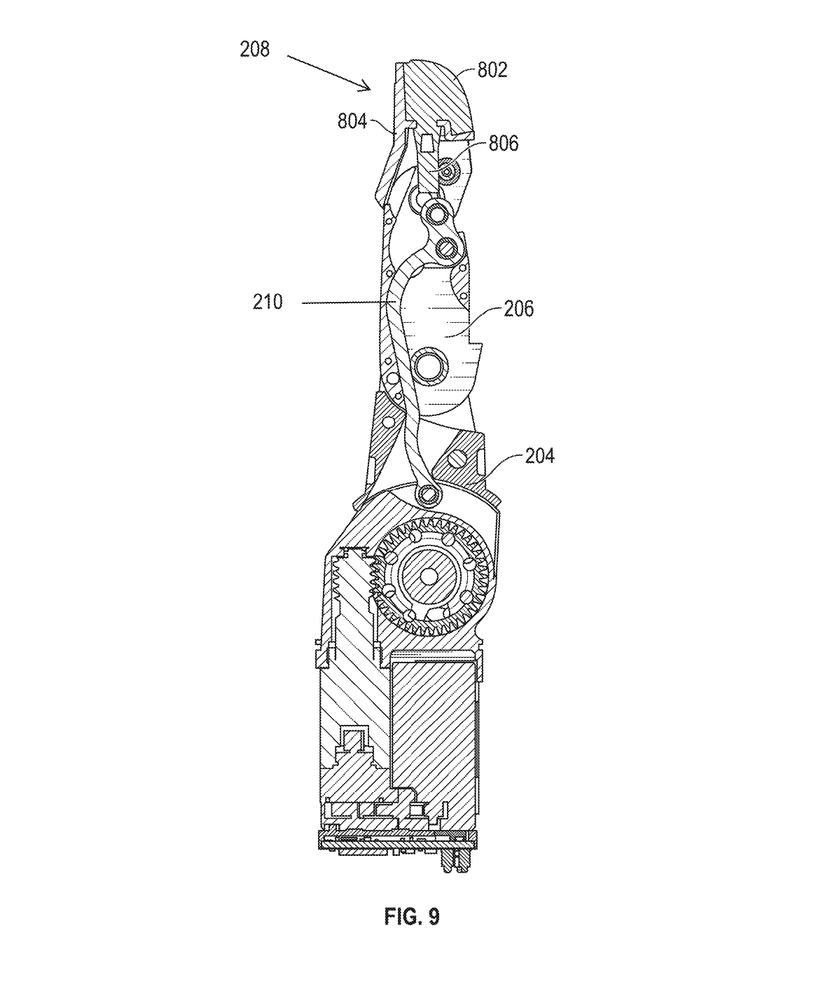

FIGS. 8A and 8B show a perspective and partially exploded view, respectively, of an example prosthetic digit 201 with a tip portion 802. The tip portion 802 may be conductive and configured to be recognized by touch screens upon contact. In some embodiments, the tip portion 802 may not be conductive. The prosthetic digit 201 may have the same or similar features and/or functions as the prosthetic digit 200 or 400, and vice versa, except as otherwise described. FIG. 9 shows a side, cross-section view of the prosthetic digit 200 taken along the line C-C as shown in FIG. 8A.

The prosthetic digit 201 can include the proximal digit segment 204, the middle digit segment 206, and the distal tip assembly 208. The proximal digit segment 204 may be formed by two lateral housings, such as the drive housing 216 and the complementary support housing 218 described above. The middle digit segment 206 may similarly be formed by two lateral housings 316, and 318. The various outer covers 230, 330 are removed to show the inside features of the digit.

Various portions of the prosthetic digit 201 may be formed of or include conductive materials. The drive housing 216 and support housing 218 of the proximal digit segment 204, the complementary lateral housings 316, 318 of the middle digit segment 206, and/or the linkage assembly 210 (that rotatably couples the proximal digit segment 204, middle digit segment 206 and distal tip assembly 208 together) may be made from a conductive, e.g. metallic, material. The distal tip assembly 208 may include a distal tip housing 804 and the tip portion 802. In some embodiments, the distal tip housing 804 may be plastic. In some embodiments, the distal tip housing 804 may be metal. The tip portion 802 may be formed from a conductive material. The tip portion 802 may be formed from a conductive elastomer. The tip portion 802 may be formed from silicone. The tip portion 802 may include a contact tail 806, which may also be formed of the conductive elastomer, such as silicone. The contact tail 806 may be an elongated, cylindrical structure that extends proximally from a proximal end of the tip portion 802. The contact tail 806 may have other shapes and extents, such as rounded, spherical, flat, stubby, etc. There may be one or multiple contact tails 806. The tip portion 802 may be configured to be pressed into the distal end of the distal tip housing 804. The tip portion 802 may be secured via friction fit. In some embodiments fasteners or other attachment may be used.

As shown assembled in FIG. 9, the contact tail 806 may contact the metallic surface of the middle digit segment 206. The contact tail 806 may in addition or alternatively contact the linkage assembly 210. The distal tip assembly 208 allows for the tip portion 802 to harness the inductance of the metallic portions of the prosthetic digit 200 such that the tip portion 802 can be used with capacitive touch screens. For example, mobile device displays, displays on tablets or in vehicles, computer screens, etc., will respond to contact from the tip portion 802 on the screen surface. In some embodiments, the tip portion 802 may not include the contact tail 806, for example where the distal tip housing 804 is made from a conductive material such as metal. In some embodiments, the tip portion 802 may be connected to or include a printed circuit board (PCB). A distal end of the tip portion 802 may be connected to the PCB board. The tip portion 802 may harness the capacitance from the PCB board rather than the metallic components of the prosthetic digit 200.

In some embodiments, the distal tip housing 804 may be made of a conductive material (e.g., a metal) and the tip portion 802 may harness the inductance of the distal tip housing 804 to allow the tip portion 802 to be used with capacitive touch screens. In such embodiments, it may not be necessary for the tip portion 802 to include the contact tail 806, as the tip portion 802 can harness sufficient inductance from the distal tip housing 804.

As discussed above, the prosthetic digit 201 may include a proximal digit cover 230 surrounding the proximal digit segment 204 and a middle segment cover 300 surrounding the middle digit segment 206. FIGS. 8A-9 show the prosthetic digit 201 with the proximal digit cover 230 and the middle segment cover 300 removed to show the metallic construction of the proximal digit segment 204 and middle digit segment 206.

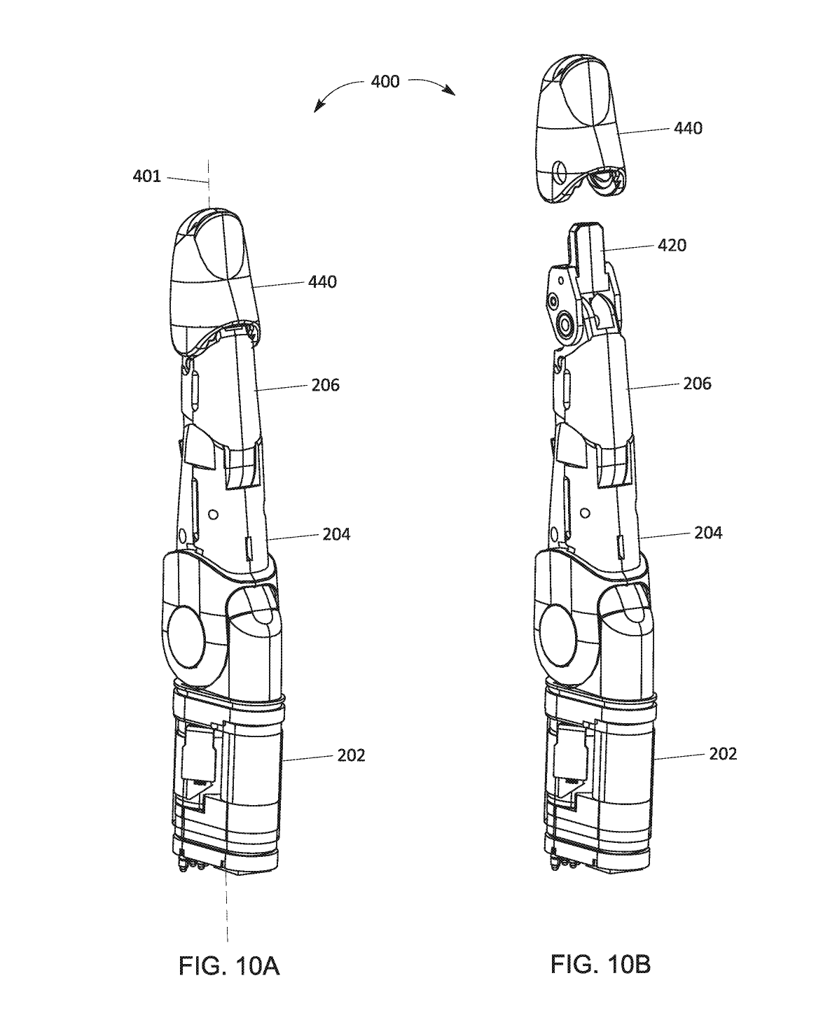

FIGS. 10A and 10B show a perspective and partially exploded view, respectively, of an example prosthetic digit 400 with a replaceable tip assembly 440. The prosthetic digit 400 may have the same or similar features and/or functions as the prosthetic digits 200, 201, and vice versa, except as otherwise described. For example, the prosthetic digit 400 can include the knuckle gearbox 202, proximal digit segment 204, and the middle digit segment 206 described above. For example, the proximal digit segment 204 may be formed by two lateral housings, such as the drive housing 216 and the complementary support housing 218 described above. The middle digit segment 206 may similarly be formed by two lateral housings 316, and 318. The various outer covers 230, 330 of the proximal digit segment 204 and middle-digit segment 206, are removed to show the inside features of the digit. Other features as described elsewhere herein may be included even if not explicitly described with respect to FIGS. 10A and 10B.

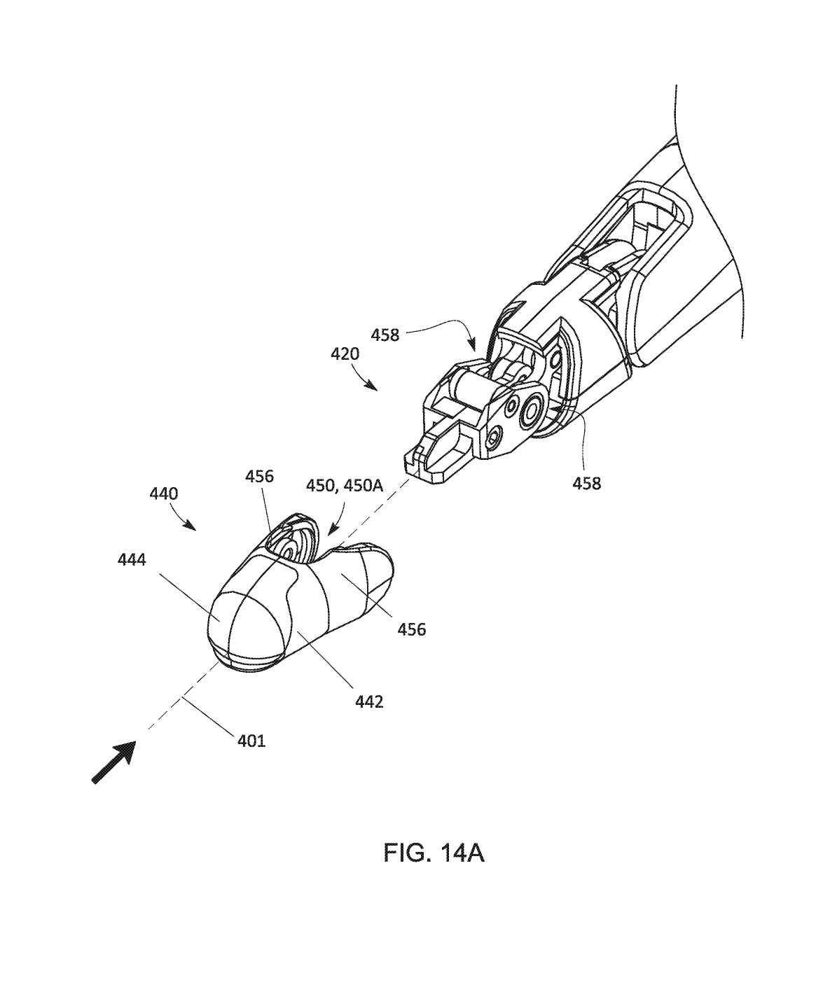

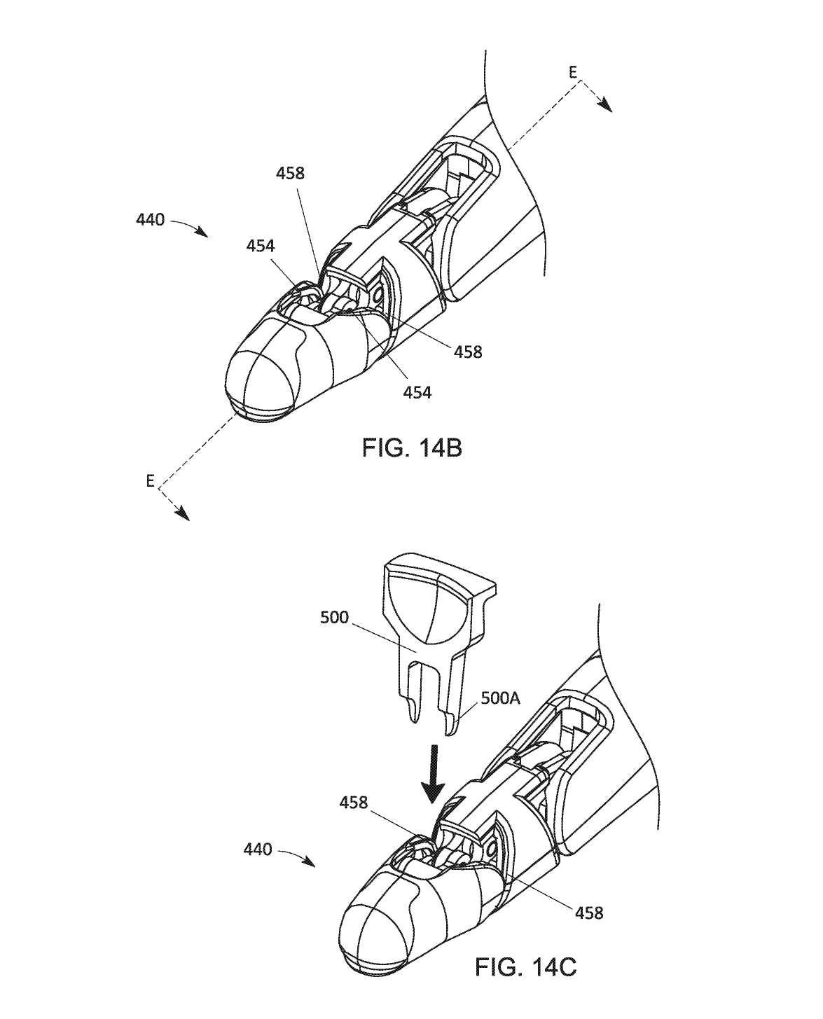

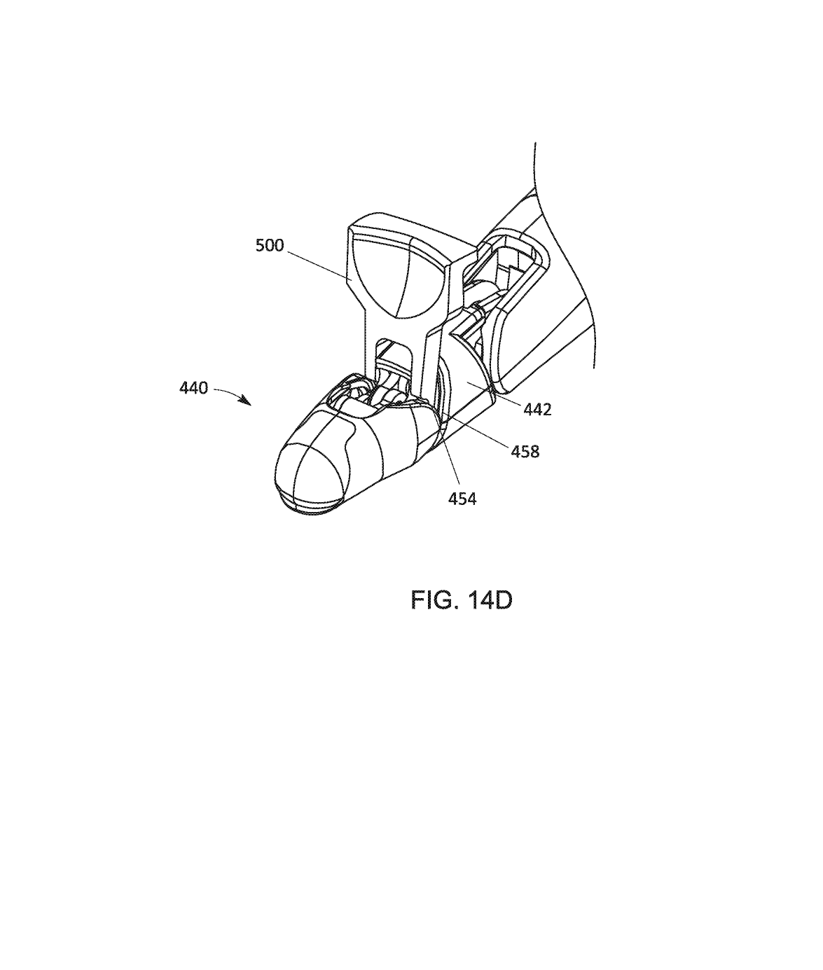

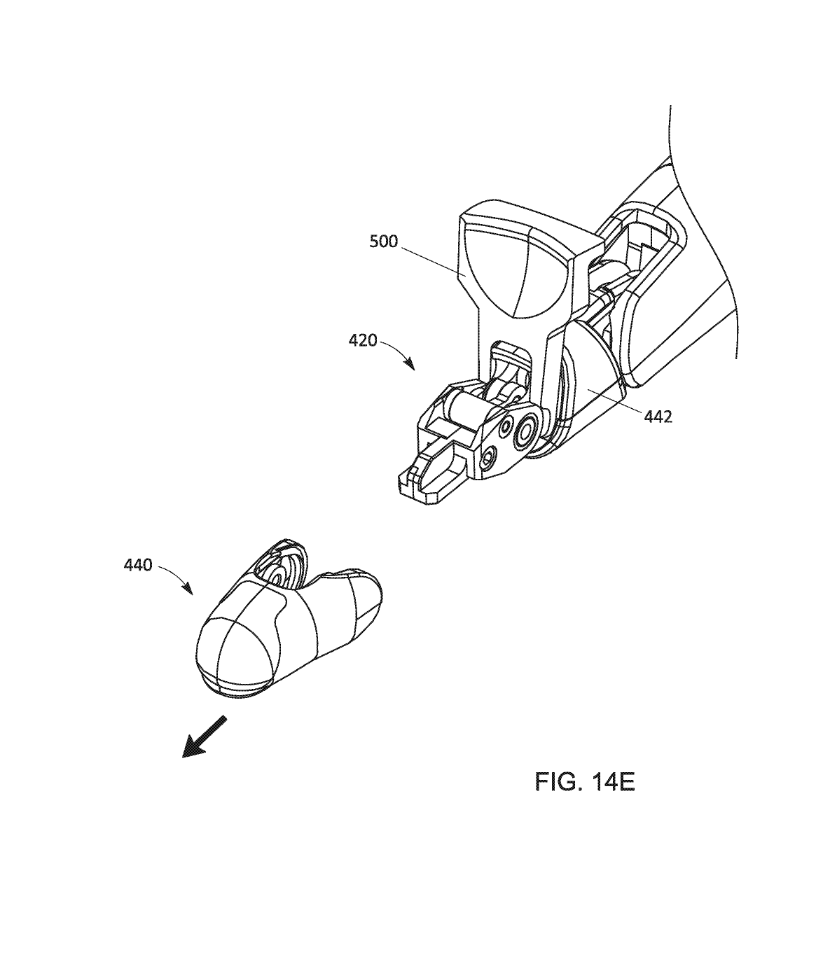

The prosthetic digit 400 may additionally include a distal phalange assembly 420. The distal phalange assembly 420 may be covered or enclosed by the replaceable tip assembly 440. The replaceable tip assembly 440 may be reversibly couplable to the distal phalange assembly 420, as described in further detail herein, for example with respect to FIGS. 14A-14E. The distal phalange assembly 420 and the replaceable tip assembly 440 may be coupled together with the knuckle gearbox 202, proximal digit segment 204, and middle-digit segment 206 to form the prosthetic digit 400. The prosthetic digit 400 may define a central axis 401 when the digit 400 is in a fully extended position, such as the configuration shown.

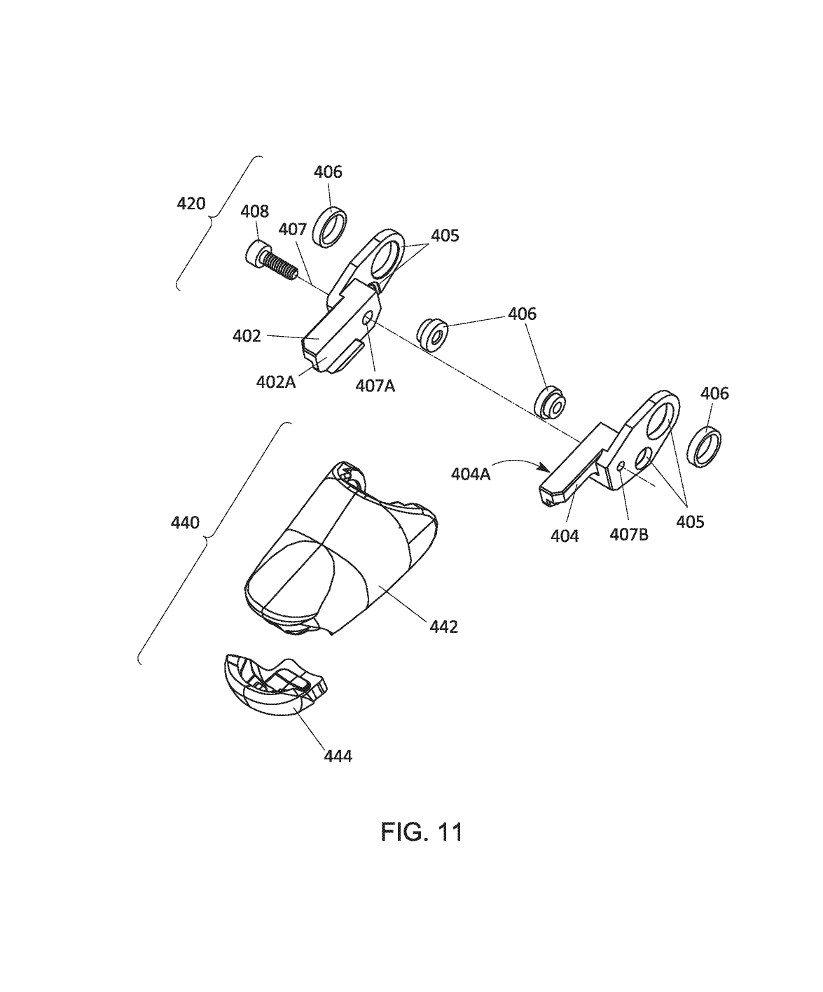

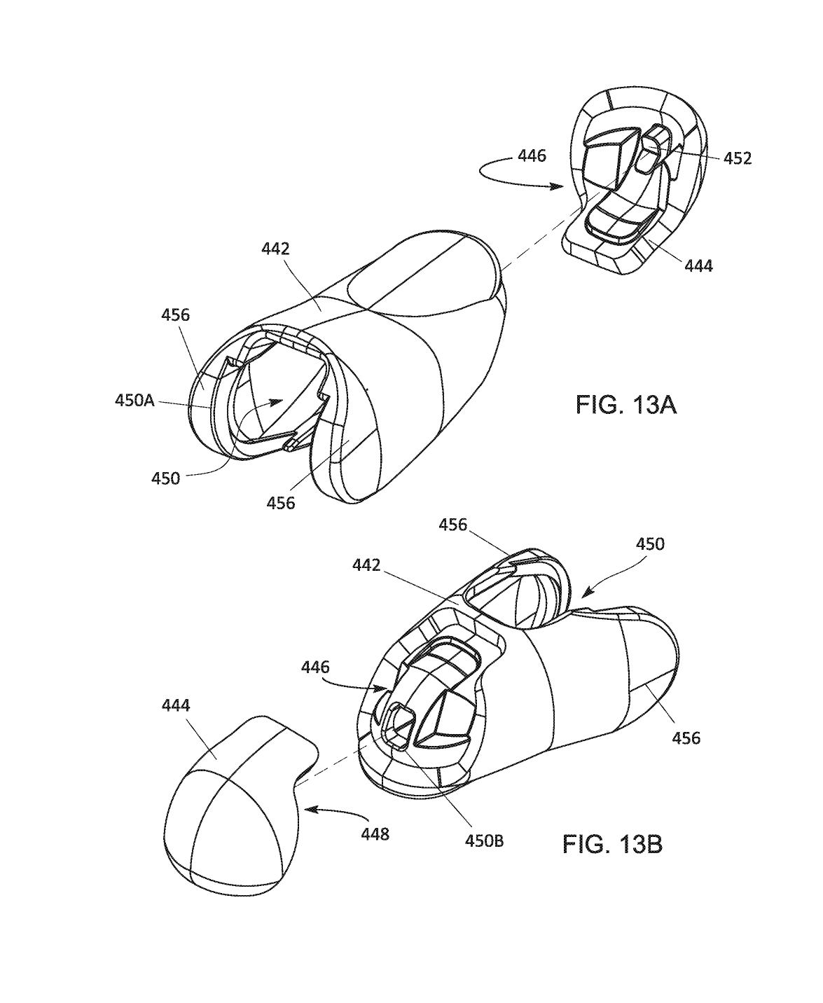

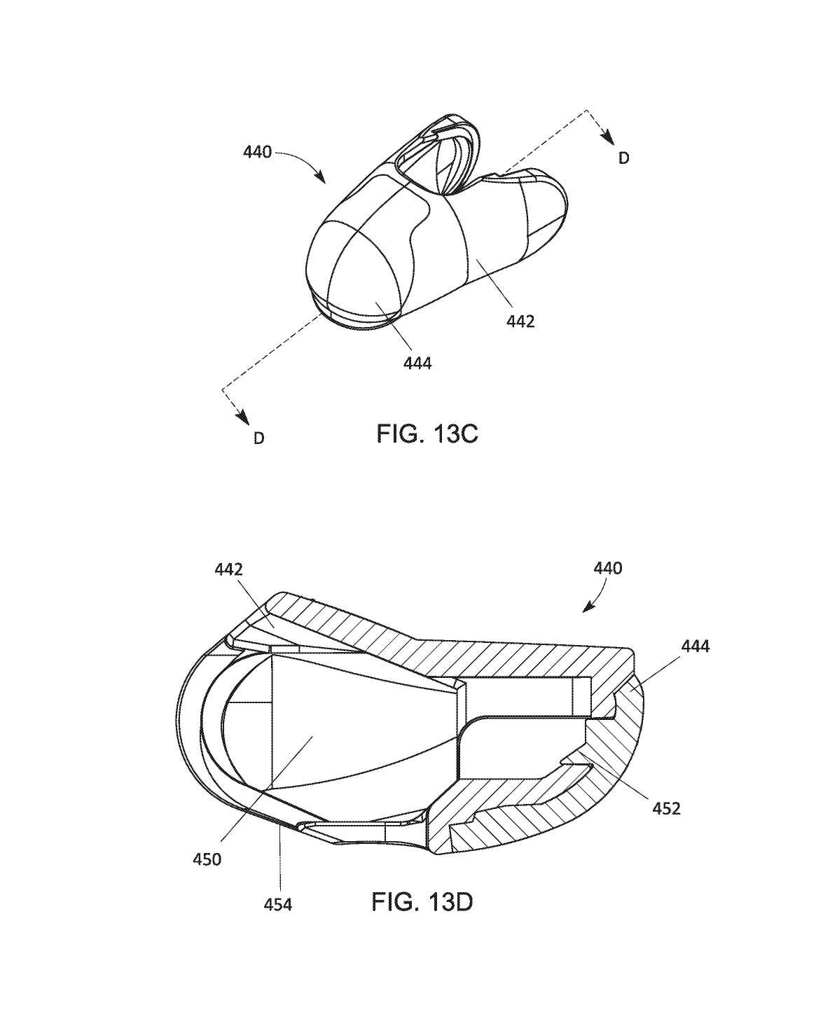

FIG. 11 is an exploded view of the replaceable tip assembly 440 and distal phalange assembly 420. The replaceable tip assembly 440 may generally include a distal cover portion 442 and an elastomer tip 444. Further details regarding the distal cover portion 442 and the elastomer tip 444 of the replaceable tip assembly 440 are described herein, for example with respect to FIGS. 13A-13D.

The distal phalange assembly 420 may be formed by two lateral housing portions 402 and 404. The lateral housing portions 402 and 404 may be constructed from a conductive, e.g. metallic, material. The lateral housing portions 402 and 404 may be constructed from aluminum. The lateral housing portions 402, 404 may be designed to join or fasten together along their respective edges (e.g., by press-fit, complementary grooves, welding, adhesives, etc.) to form the distal phalange of the prosthetic digit 400. An inner-facing surface 402A of the housing portion 402 may include mating features (e.g., protrusions, recesses, complementary grooves, etc.) that correspond with complementary mating features on the inner surface 404A of the housing portion 404, and vice versa. The constructions of the lateral housing portions 402 and 404 may ensure and/or facilitate proper alignment of the housing portion 402 and the housing portion 404 when they are assembled together.

The lateral housing portions 402 and 404 may be further secured or retained to each other by way of a fastener such as a screw 408 as shown. In some examples, the screw 408 may be a cap head screw with a diameter between approximately 1 mm and approximately 2 mm. There may be a securement lumen extending along an axis 407 through the distal phalange assembly 420 and configured to receive the screw 408. For example, the housing portion 402 may define an opening 407A, and the housing portion 404 may define an opening 407B, which axially align to form the securement lumen along the axis 407 when the housing portions 402 and 404 are assembled together. The openings 407A and 407B of the housing portions 402 and 404 may be threaded, to allow the securement lumen along the axis 407 to engage with complementary threads of the screw 408, or not threaded. In some examples, the housing portions 402 and 404 may additionally or alternatively be secured to each other by other suitable means (e.g., welding, gluing, taping, etc.).

Each of the housing portions 402, 404 may include one or more attachment openings 405 to facilitate attaching the distal phalange assembly 420 to the other components of the prosthetic digit 400. The attachment openings 405 may facilitate rotation of the distal phalange assembly. Each of the attachment openings 405 may form a lumen or through-hole in the bodies of their respective housing portion.

Each attachment opening 405 of the housing portion 402 may have features that correspond to and axially align with a respective attachment opening 405 of the housing portion 404, and vice versa. The housing portion 402 and the housing portion 404 may have the same number of attachment openings 405. For example, the housing portions 402 and 404 may each have one, two, or three, attachment openings 405.

In examples where the housing portions 402 and 404 have multiple attachment openings 405, each of the attachment openings 405 may have a different size. In other examples where the housing portions 402 and 404 have multiple attachment openings 405, all of the attachment openings 405 may be the same size. The attachment openings 405 may have a circular and/or rounded shape.

The locations of the attachment openings 405 of the housing portion 402 may mirror the locations of the attachment openings 405 of the housing portion 404, and vice versa. Each attachment opening 405 of the housing portion 402 may axially align with a corresponding attachment opening 405 on the housing portions 404 when the distal phalange assembly 420 is assembled to axially receive therethrough or therein other components.

The attachment openings 405 may each include a bushing 406. The bushings 406 may be made from a strong, wear-resistant material that is suitable for heavy loads. For example, the bushings 406 may be made from a metallic material such as bronze, brass, steel, titanium, etc. The bushings 406 may be step bushings. Each bushing may be sized to fit tightly against the inside luminal surface of its respective attachment opening 405. The bushings 406 may be press-fit into the attachment openings 405 of the housing portions 402, 404 prior to the housing portions 402, 404 being assembled and secured together. The bushings 406 may be bonded within the attachment openings 405 of the housing portions 402, 404 prior to the housing portions 402, 404 being assembled and secured together. The bushings 406 may be ring-shaped. The bushings 406 may have a ring-shape with a stepped-down diameter axial protrusion or lip.

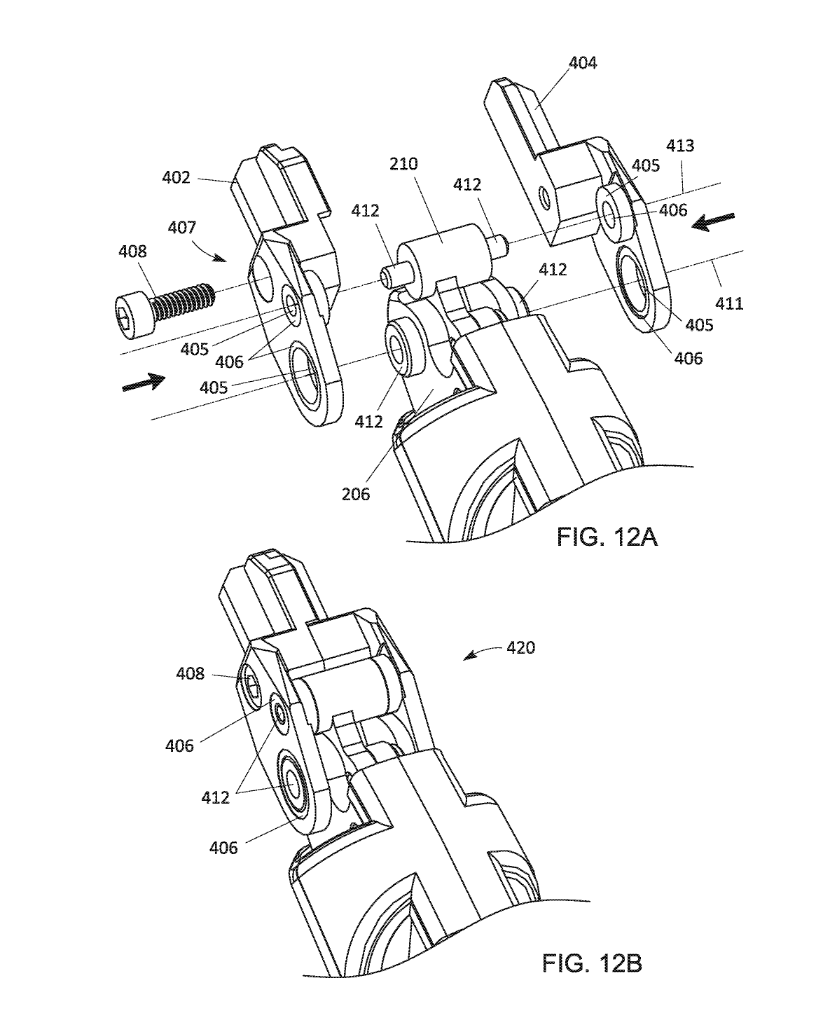

FIGS. 12A and 12B show exploded and assembled views, respectively, of how the distal phalange assembly 420 may be attached to the remainder of the prosthetic digit 400. The prosthetic digit 400 may include complementary mating features for engaging with the attachment openings 405 of the lateral housing portions 402 and 404. The prosthetic digit 400 may have one or more pivot bosses 412 for engaging with the attachment openings 405 of the housing portions 402 and 404. The pivot bosses 412 may be configured to rotatably couple the distal phalange assembly 420 to the middle digit segment 206 and the linkage assembly 210 along dorsally and/or distally offset axes 411, 413 as shown.

There may be a pivot boss 412 located on either or both lateral sides of the middle digit segment 206. The pivot bosses 412 of the middle digit segment 206 may define a first pivot axis 411. The first pivot axis 411 may be positioned at or near the distal end of the middle digit segment 206. There may be a boss 412 located on either lateral side of the distal end of the linkage assembly 210. The pivot bosses 412 of the linkage assembly 210 may define a second pivot axis 413. The second pivot axis 413 may be positioned at or near the distal end of the linkage assembly 210. The pivot axes 411, 413 may be spaced distally and/or or dorsally from each other. The pivot axis 413 may be located distal from and dorsal to the pivot axis 411 when the tip is in the straight configuration.

The pivot bosses 412 may be cylindrical protrusions with a circular cross-section. The pivot bosses 412 may extend laterally outward from the central axis 401. The number of pivot bosses 412 on the prosthetic digit 400 may be the same as the number of attachment openings 405 of the housing portions 402 and 404. The pivot bosses 412 may be configured to mate or engage with the inner surfaces of the bushings 406 within the attachment openings 405 of the housing portions 402 and 404. The outer dimensions of pivot bosses 412 may be slightly smaller than inner dimensions of the bushings 406 such that the pivot bosses 412 can be received within the bushings 406.

To attach the distal phalange assembly 420 to the prosthetic digit 400, the housing portion 402 and the housing portion 404 may be placed or positioned at parallel locations on either lateral side of the prosthetic digit 400, as shown in FIG. 12A. The housing portions 402 and 404 may be positioned such that their respective inner-facing surfaces 402A and 404A face towards each other and perpendicular to the central axis 401 of the prosthetic digit 400. The housing portions 402, 404 may be positioned such that each of the attachment openings 405 are aligned with a corresponding pivot boss 412. The housing portions 402, 404 may each have an attachment opening 405 aligned along the first pivot axis 411 which may be defined by the pivot bosses 412 on the middle digit segment 206. The housing portions 402, 404 may each have an attachment opening 405 aligned along the second pivot axis 413 which may be defined by the pivot bosses 412 on the linkage assembly 210.

The two housing portions 402, 404 may then be pushed or press-fitted together (e.g., pushed together in the directions indicated by the arrows in FIG. 12A) to form the distal phalange assembly 420. As discussed above, a screw 408 or other fastener may then be inserted within the lumen along the axis 407 to further secure the housing portions 402, 404 of distal phalange assembly 420 together.

As shown in FIG. 12B, the pivot bosses 412 are received within the bushings 406 and attachment openings 405 of the two lateral housing portions 402, 404 when the housing portions 402, 404 are assembled together. The pivot bosses 409 may be able to rotate within the bushings 406 of the first pivot axis 411 and second pivot axis 413 to pivot inwards when the digit is driven to close and outwards when the digit is driven to open.

FIGS. 13A-D show various views of the replaceable tip assembly 440 that may be used with the prosthetic digit 400. As discussed above with respect to FIG. 11, the replaceable tip assembly 440 may generally include a distal cover portion 442 and an elastomer tip 444. The replaceable tip assembly 440 may function as a cover for the distal phalange assembly 420 of the prosthetic digit 400. The replaceable tip assembly 440 may be removably couplable with the distal phalange assembly 420 of the prosthetic digit 400, as discussed further below with respect to FIGS. 14A-14E.

As shown in FIGS. 13A-D, the distal cover portion 442 may be sized and shaped to surround or enclose the distal phalange assembly 420. The distal cover portion 442 may be a hollow body. The body of the distal cover portion 442 may define an internal cavity 450 having a proximal opening 450A and a distal opening 450B. The distal cover portion 442 may be generally rigid while retaining some flexibility. The distal cover portion 442 may be capable of deforming (e.g. flexing) to a widened configuration in response to an external force and returning to a neutral configuration once the external force is removed. The distal cover portion 442 may be constructed from a plastic material. The distal cover portion 442 may be constructed from a thermoplastic material. The distal cover portion 442 may be constructed from a Polycarbonate-Acrylonitrile Butadiene Styrene (“PC/ABS”) material.

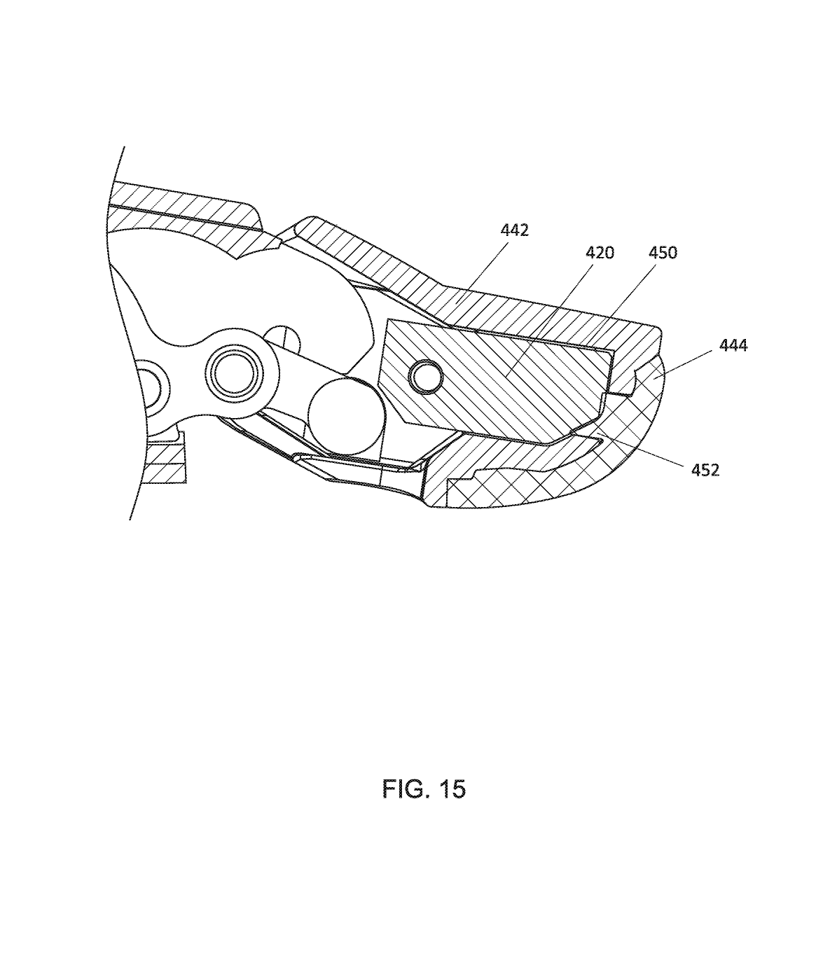

The elastomer tip 444 may be configured to aid the gripping capabilities of the prosthetic digit 400. The elastomer tip 444 may be constructed from a compliant material. The elastomer tip 444 may be constructed from a material with a high coefficient of friction. The elastomer tip 444 may be constructed from a silicon material. In some examples, the elastomer tip 444 may be constructed from a conductive elastomer to allow for touch-screen compatibility, as discussed further below with respect to FIG. 15. In some examples, the elastomer tip 444 may not be conductive.

The distal cover portion 442 and the elastomer tip 444 may be bonded together to form the replaceable tip assembly 440. The distal cover portion 442 may have a mating surface 446 configured to engage with a mating surface 448 of the elastomer tip 444. As shown in FIGS. 13A-B, the mating surface 446 of the distal cover portion 442 may have a contour and/or mating features (e.g., protrusions, recesses, complementary grooves, etc.) that correspond with complementary contour and/or mating features on the mating surface 448 of the elastomer tip 444. The mating surface 446 and the mating surface 448 may have even molding depths to reduce likelihood of sink. The mating surface 446 and the mating surface 448 may include multiple bonding surfaces to provide a secure location of the elastomer tip 444 within the distal cover portion 442.

The mating surface 448 of the elastomer tip 444 may include a protrusion or extended boss 452 extending inwardly and/or proximally. The extended boss 452 may be sized and shaped to correspond with the size and shape of the distal opening 450B of the internal cavity 450 of the distal cover portion 442. The extended boss 452 of the elastomer tip 444 may be pressed into the distal opening 450B of the distal cover portion 442 to form the assembled replaceable tip assembly 440 as shown in FIG. 13C. The extended boss 452 may be secured via friction fit. As shown in the cross-section view of FIG. 13D (taken across line D-D in FIG. 13C and, for clarity, flipped vertically), the extended boss 452 may protrude through the body of the distal cover portion 442 into the internal cavity 450 when the replaceable tip assembly 440 is assembled.

As discussed above, the replaceable tip assembly 440 may be removably couplable to the prosthetic digit 400. The replaceable tip assembly 440 may be removed manually or using a simple tool. This feature advantageously allows a user to repair and/or replace a replaceable tip assembly 440 that has been damaged, without having to remove other components of the digit 400.

In some examples, the replaceable tip assembly 440 may be removably couplable to the distal phalange assembly 420. A proximal end of the distal cover portion 442 may be configured to engage or mate with the distal phalange assembly 420. The proximal end of the distal cover portion 442 may include a pair of proximally-extending lugs 456 (see FIG. 13B). The lugs 456 may have a shape corresponding to the shape of the lateral housing portions 402 and 404 of the distal phalange assembly 420. The lugs 456 may be designed to attach to a pair of connection surfaces 458 at a proximal end of the distal phalange assembly 420, as further described. As shown in FIG. 13D, a ventral or bottom surface of the lugs 456 may have an angled face 454. The angled face 454 may facilitate the removal or un-coupling of the replaceable tip assembly 440 from the distal phalange assembly 420.

FIGS. 14A-14E illustrate how the replaceable tip assembly 440 may reversibly couple to the prosthetic digit 400. FIGS. 14A and 14B illustrate how the replaceable tip assembly 440 may couple with the distal phalange assembly 420 to form the prosthetic digit 400. As shown in FIG. 14A, the replaceable tip assembly 440 and the distal phalange assembly 420 may be aligned together along the central axis 401 of the prosthetic digit 400. The central axis 401 may be defined by the prosthetic digit 400 in the straightened or fully extended configuration. The distal phalange assembly 420 and replaceable tip assembly 440 may then be pushed or joined axially together while maintaining alignment with the central axis 401. The distal phalange assembly 420 may be received by the internal cavity 450 of the replaceable tip assembly 440 through the proximal opening 450A. The lugs 456 may displace (e.g. flex) outwards as the proximal end of the replaceable tip assembly 440 travels proximally over distal portions of the distal phalange assembly 420.

The lugs 456 may snap back inwards once they are aligned with connection surfaces 458 on the distal phalange assembly 420. The connection surfaces 458 may be located at the proximal ends of the lateral housing portions 402 and 404. As discussed above, the size and shape of the lugs 456 may correspond to the size and shape of the proximal ends of the lateral housing portions 402, 404 of the distal phalange assembly 420, such that the lugs 456 snap or clip on to the proximal ends of the lateral housing portions 402, 404 of the distal phalange assembly 420, as shown in FIG. 14B. An inner surface or contour defined by the inner-facing sides of the lugs 456 may surround the outer surface or contour defined by the connection surfaces 458. The connection surfaces 458 may include outer surfaces or contours of distal portions of the lateral housing portions 402, which may surround respective attachment openings 405.

FIGS. 14C-E show how the replaceable tip assembly 440 may be un-coupled (e.g. separated) from the distal phalange assembly 420. In some examples, a removal tool 500 may be used to un-couple the replaceable tip assembly 440 from the distal phalange assembly 420. The removal tool 500 may have a working end 500A that can be contoured and corresponds to the contour of the angled face 454 of the lugs 456. The working end 500A may include two prongs each having respective contours at ends thereof. A user may align the working end 500A of the removal tool 500 with the angled face 454 of the lugs 456, on a dorsal side of the digit, as shown in FIG. 14C.

As shown in FIG. 14D, the removal tool 500 may then be pressed in the ventral direction firmly against the angled face 454 at the proximal end of the replaceable tip assembly 440. The working end 500A of the removal tool may engage and flex the distal cover portion 442 to release the tip assembly 440 from the distal phalange assembly 420. The contour of the working end 500A of the removal tool 500 may interface with the angled face 454 on the proximal end of the replaceable tip assembly 440 such that it pries or separates the lugs 456 from around the proximal ends of the lateral housing portions 402 and 404 of the distal phalange assembly 420. The lugs 456 may flex outwardly to outward lateral positions beyond the connection surfaces 458 on the distal phalange assembly 420. The replaceable tip assembly 440 may then be slid distally along and off of the distal phalange assembly 420 as shown in FIG. 14E.

To re-attach the tip assembly 440, or to attach a new tip assembly 440, the process may be repeated beginning as described in FIG. 14A. The tip assembly 440 may be slid onto and snapped into place, as described. In some embodiments, the removal tool 500 may also be used to assist with securing the tip assembly 440 onto the digit, in the reverse manner, as described in FIG. 14E and then FIG. 14D.

FIG. 15 is a cross-sectional view of the prosthetic digit 400 taken across the line E-E in FIG. 14B and, for clarity, flipped vertically. As shown, the distal phalange assembly 420 is located within the replaceable tip assembly 440. As discussed above, the distal phalange assembly 420 may be housed within the internal cavity 450 of the distal cover portion 442 when the prosthetic digit 400 is assembled. The distal phalange assembly 420 may fill all or most of the internal cavity 450. As shown, the extended boss 452 of the elastomer tip 444 may contact the distal phalange assembly 420 within the internal cavity 450. The extended boss 452 may make compressive contact with the distal phalange assembly 420.

In some embodiments, the contact between the extended boss 452 and the distal phalange assembly 420 may facilitate a conductive path between the elastomer tip 444 and the distal phalange assembly 420. The contact between the extended boss 452 and the distal phalange assembly 420 may allow the elastomer tip 444 to harness the inductance of the metallic portions of the prosthetic digit 400 such that the elastomer tip 444 can be used with capacitive touch screens. For example, mobile device displays, displays on tablets or in vehicles, computer screens, etc., will respond to contact from the elastomer tip 444 on the screen surface. In some embodiments, the extended boss 452 may contact other metallic or conductive portions of the prosthetic digit 400, such as the linkage assembly 210. Further, the elastomer tip 444 can be used with capacitive touch screens without requiring any physical connection between the elastomer tip 444 and the user’s body. Thus there need not be a conductive path from the elastomer tip 444 to the body of a human user.

### Embodiments and Terminology

Various modifications to the implementations described in this disclosure can be readily apparent to those skilled in the art, and the generic principles defined herein can be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results, except as otherwise described. In some cases, the actions recited in the claims can be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. A prosthetic hand comprising:
a palm portion;
a prosthetic digit comprising a proximal digit segment, the proximal digit segment comprising a drive housing and a support housing, respective edges of the drive housing and the support housing configured to couple together to form the proximal digit segment;
a knuckle attachment portion configured to couple with the proximal prosthetic digit segment and the palm portion, the knuckle attachment portion comprising:
a lip seal having a cross-sectional shape comprising a main body portion and a contact finger extending away from the main body portion;
a knuckle gearbox; and
a bearing shaft; and
an axis defined by the drive housing, the knuckle attachment portion, and the support housing, wherein the bearing shaft is configured to extend along the axis to couple the drive housing and the support housing to the knuckle gearbox, and wherein the lip seal is configured to provide a waterproof seal between the knuckle gearbox and the drive housing.

2. The prosthetic hand of claim 1, wherein the contact finger comprises a plateaued portion and a wiper corner.

3. The prosthetic hand of claim 1, wherein the contact finger is configured to deflect toward the main body portion.

4. The prosthetic hand of claim 1, wherein the contact finger is configured to deflect away from the main body portion.

5. The prosthetic hand of claim 1, further comprising a waterproof cap to seal the proximal digit segment at the drive housing.

6. The prosthetic hand of claim 1, wherein an end of the drive shaft comprises a flat top configured to seal the proximal digit segment at the support housing.

7. The prosthetic hand of claim 1, further comprising a support seal configured to provide a waterproof seal between the knuckle gearbox and the support housing.

8. The prosthetic hand of claim 7, wherein the support seal is an O-ring.

9. The prosthetic hand of claim 1, wherein the knuckle gearbox is configured to house a worm wheel.

10. The prosthetic hand of claim 1, wherein an internal surface of the drive housing comprises a protrusion defining a groove configured to receive the lip seal.

11. The prosthetic hand of claim 1, further comprising a proximal digit cover disposed over the drive housing and the support housing of the proximal digit segment.

12. A prosthetic hand comprising:
a prosthetic digit comprising a proximal digit segment;
a knuckle gearbox rotatably coupled with the proximal digit segment about an axis of rotation; and
a lip seal extending annularly around the axis of rotation between and contacting both the knuckle gearbox and the proximal digit, the lip seal having a cross-sectional shape comprising a main body portion and a contact finger extending radially outwardly away from the main body portion.

13. The prosthetic hand of claim 12, wherein the contact finger comprises a plateaued portion and a wiper corner, wherein the plateaued portion of the contact finger has a width greater than or equal to a width of the main body portion.

14. The prosthetic hand of claim 12, wherein the contact finger comprises a plateaued portion and a wiper corner, wherein the plateaued portion of the contact finger has a width less than a width of the main body portion.

15. The prosthetic hand of claim 12, wherein the contact finger is configured to deflect relative to the main body portion to provide a constant amount of interference between the seal and the knuckle gearbox.

16. The prosthetic hand of claim 12, wherein the main body portion comprises a long dimension L that extends parallel to the axis of rotation and a short dimension W that extends perpendicular relative to the axis of rotation.

17. A lip seal for a prosthetic digit, the lip seal comprising:
a main body portion extending annularly along a circumferential path and having a cross-sectional profile with a first dimension configured to extend parallel to an axis of rotation when assembled with the prosthetic digit; and
a contact finger extending annularly along the circumferential path and having a cross-sectional profile that is inclined outwardly from the main body portion, wherein the contact finger is configured to deflect relative to the main body portion to provide a constant amount of interference between the seal and a rotatable component of the prosthetic digit.

18. The lip seal of claim 17, wherein the contact finger comprises a plateaued portion, wherein the plateaued portion of the contact finger has a width greater than or equal to a width of the main body portion.

19. The lip seal of claim 17, wherein the contact finger comprises a plateaued portion, wherein the plateaued portion of the contact finger has a width less than a width of the main body portion.

20. The lip seal of claim 17, wherein the contact finger and the main body portion form a cross-sectional profile in the shape of a “V” or a canted-V.
